(19) United States
(12) Patent Application Publication
(10) Pub. No.: US 2008/0108128 A1
(43) Pub. Date: May 8, 2008

(54) Resonance Energy Transfer Assay System for Multi-Component Detection

(76) Inventors: Karin A. Eidne, Western Australia (AU); Karen M. Kroeger, Western Australia (AU); Ulrich Schmidt, Western Australis (AU)

Correspondence Address: FISH & RICHARDSON P.C., PO BOX 1022, MINNEAPOLIS, MN, 55440-1022, US

(21) Appl. No.: 10/580,130

(22) PCT Filed: Nov. 18, 2004
(86) PCT No.: PCT/AU04/01598

§ 371 Date: May 19, 2006

(30) Foreign Application Priority Data
Nov. 19, 2003 (AU) 2003906398

(51) Int. Cl. C12M 1/00; G01N 21/64; C12N 15/11; C12N 15/00; C12N 5/06; C12N 5/04; C12N 1/20; C12N 7/00; C12N 1/00

(52) U.S. Cl. 435/235.1; 250/221; 435/288.7; 435/320.1; 536/23.4; 435/325; 435/348; 435/366; 435/410; 435/252.3; 435/254.2

(57) ABSTRACT

The present invention relates to a system for detecting molecular associations. In particular, the present invention relates to a multi-component detection system comprising: i). a first agent comprising a first interacting group coupled directly or indirectly to a first tag, which first tag emits light of a first wavelength upon activation by a substrate or energy source, which produces a first activated tag; ii). a second agent comprising a second interacting group coupled directly or indirectly to a second tag, which second tag can accept the energy from the first tag when the first and second interacting groups are associated and an appropriate substrate or energy source for the first tag is present thereby producing a second activated tag that emits light of a second wavelength; iii). a third agent comprising a third interacting group coupled directly or indirectly to a third tag that can accept the energy from the first activated tag when the first and third interacting groups are associated and an appropriate substrate or energy source for the first tag is present to produce a third activated tag that emits light of a third wavelength; iv). an appropriate substrate or energy source to activate the first tag, and v). a means of detecting said emitted light.

### FIELD OF THE INVENTION

The present invention relates to a system for detecting molecular associations. In particular, the present invention relates to a multi-component detection system, wherein the molecular association of two or more components is detected.

### BACKGROUND OF THE INVENTION

In the post genomic era proteomics has become more and more important. It includes the identification of all proteins encoded by the genome that are expressed in a cell, and the description of their behaviour, including expression, interactions and function.

Proteins do not act in isolation in a cell, but rather in stable or transitory complexes, with protein-protein interactions being key determinants of protein function (Auerbach et al., (2002), Proteomics, 2, 611-623). Furthermore, proteins and protein complexes interact with other cellular components like DNA, RNA and small molecules. Unravelling and dissecting out individual proteins involved in these interactions is crucial for the understanding of biological processes.

To this end a number of assay techniques have been developed over the years to assist in determining biological interactions. However, many of these techniques are either not suitable for high throughput screening or involve costly procedures. For example, techniques such as co-immunoprecipitation, have been used for many years to validate protein-protein interactions; however, this technique is not amenable to automation or high throughput screening. Other techniques such as co-immunoprecipitation combined with mass spectrometry (Anderson & Mann, (2000), FEBS Lett, 480, 25-31) is too complex, time consuming and expensive to be of use in drug screening programs. Surface plasmon resonance (SPR) is a highly sensitive and accurate technique capable of detecting biological interactions. However, this technique requires sufficient quantities of the purified target protein to be immobilised on the sensor surface and does not yield any information on the identity of ligands that may bind to it in a complex mixture of molecules.

Other techniques that have been developed include AlphaScreen System™, which is highly sensitive and versatile, but requires the interacting molecules to be available in a purified state; fluorescence polarisation and fluorescence anisotropy (Pope et al., (1999), Drug Disc Today, 4, 350-362), which is useful in high-throughput screening, but produces a number of false results and the dynamic range is limited; and fluorescence correlation spectroscopy (Pope et al., (1999), Drug Disc Today, 4, 350-362), which has a wide dynamic range, but the mass difference between the interacting partners must be large and the analysis is complex.

More importantly, all the above methods share the major disadvantage that the detection only occurs in vitro. This artificial situation does often not accurately reflect the intracellular environment where proteins interact and ‘cross-talk’ with many different partners. Also, interactions depend on buffer conditions and interactions may be abolished or initiated by the choice of inappropriate conditions, thus increasing the number and likelihood of false positive and false negative results.

As a consequence, a number of detection systems have been developed to detect protein-protein interactions ‘in vivo’. For example, the yeast 2-hybrid system (Fields & Song, (1989), Nature, 340, 245-246) has been widely used. However, this technique is only capable of monitoring protein-protein interactions inside the nucleus of living yeast cells. Therefore, the important class of membrane proteins and post-translational modifications specific to mammalian cells cannot be analysed.

Fluorescence resonance energy transfer (FRET) is another detection system capable of detecting in vivo protein-protein reactions (Forster, (1948), Ann. Phys. 2, 57-75). This technique became particularly attractive and applicable to assays in living cells when the green fluorescent protein (GFP) and its mutant variants with different spectral characteristics were cloned. This allowed the genetic attachment of GFP and its variants to any target protein by fusing the encoding DNA sequences (Heim et al., (1994), PNAS. USA. 91, 12501-12504). Compared to the yeast 2-hybrid system, FRET has the advantage that the monitored interactions can occur anywhere inside the cell. FRET can be determined in any cell type (mammalian, yeast, bacterial etc.) or cell-free system. It can be detected by fluorescence spectroscopy; fluorescence microscopy and fluorescence activated cell sorting (FACS). However, as discussed below, FRET has one major drawback, it can only be used to detect a single interaction.

Bioluminescence resonance energy transfer (BRET) is another technique that has been developed to study in vivo protein-protein interactions/reactions (Xu et al., (1999), PNAS. USA, 96, 151-156; Eidne et al., (2002), Trends Endocrin. Metabol. 13, 415-421). Similar to FRET, this technology has the advantage that the detection occurs within living cells and is not restricted to a particular cellular compartment. Additionally, it overcomes several potential limitations of FRET: as the light is generated intrinsically by the luciferase, the detection system does not need to discriminate between the comparably weak signal resulting from the resonance energy transfer and the strong excitation light source. Furthermore, photo bleaching of the fluorophores and autofluorescence of the cells is not observed.

However, a major limitation of BRET, like FRET, is that only single, one-to-one interactions can be detected. However, it is widely accepted that most proteins have many more than one potential binding partner. Others act in larger complexes of two or more, and the function of a particular protein can critically depend on the presence of other proteins in the complex. Thus, looking at a single interaction does not address aspects of multiple functionality, specificity and cross-reactivity of a particular protein.

Consequently, there is a need for a multi-component detection system, which is capable of detecting multiple protein-protein associations in vitro and in vivo. More importantly, there is a need for a system that can analyse multiple associations in parallel, thus increasing throughput and reducing time and costs and a system that can analyse proteins and other biologically relevant molecules as part of multi-component molecular associates.

### SUMMARY OF THE INVENTION

Inventors have now developed a multi-component detection system, which is capable of overcoming or at least alleviating some of the problems identified in the prior art systems, while still being capable of detecting multiple interactions in vitro and in vivo.

Accordingly, in a first aspect there is provided a multi-component detection system comprising:

i). a first agent comprising a first interacting group coupled directly or indirectly to a first tag, which first tag emits light of a first wavelength upon activation by a substrate or energy source, which produces a first activated tag;

ii). a second agent comprising a second interacting group coupled directly or indirectly to a second tag, which second tag can accept the energy from the first tag when the first and second interacting groups are associated and an appropriate substrate or energy source for the first tag is present thereby producing a second activated tag that emits light of a second wavelength;

iii). a third agent comprising a third interacting group coupled directly or indirectly to a third tag that can accept the energy from the first activated tag when the first and third interacting groups are associated and an appropriate substrate or energy source for the first tag is present to produce a third activated tag that emits light of a third wavelength;

iv). an appropriate substrate or energy source to activate the first tag, and

v). a means of detecting said emitted light.

In a second aspect there is provided a multi-component detection system comprising:

i). a first agent comprising a first interacting group coupled directly or indirectly to a first tag, which first tag emits light of a first wavelength upon activation by a substrate or energy source which produces a first activated tag;

ii). a second agent comprising a second interacting group coupled directly or indirectly to a second tag, which second tag can accept the energy from the first tag in i) when the first and second interacting groups are associated and an appropriate substrate or energy source for the first tag in i) is present thereby producing a second activated tag that emits light of a second wavelength;

iii). a third agent comprising a third interacting group coupled directly or indirectly to a third tag that can accept the energy from the second activated tag in ii) when the first, second and third interacting groups are associated and an appropriate substrate or energy source for the first tag in i) is present to produce a third activated tag that emits light of a third wavelength, but said third tag is not substantially activated by the first activated tag in i) when only the first and third interacting groups are associated;

iv). an appropriate substrate or energy source to activate the tag in i); and

v). a means of detecting said emitted light.

In a third aspect there is provided a multi-component detection system comprising:

i). a first agent comprising a first interacting group coupled directly or indirectly to a first tag, which first tag emits light of a first wavelength upon activation by a substrate or energy source which produces a first activated tag;

ii). a second agent comprising a second interacting group coupled directly or indirectly to a second tag, which second tag can accept the energy from the first tag in i) when the first and second interacting groups are associated and an appropriate substrate or energy source for the first tag in i) is present thereby producing a second activated tag that emits light of a second wavelength;

iii). a third agent comprising a third interacting group coupled directly or indirectly to a third tag that can accept the energy from the first activated tag in i) when the first and third interacting groups are associated and an appropriate substrate or energy source for the first tag in i) is present and that can accept the energy from the second activated tag in ii) when the second and third interacting groups are associated and an appropriate substrate or energy source for the second tag in ii) is present to produce a third activated tag that emits light of a third wavelength;

iv). an appropriate substrate or energy source to activate the tags in i) and ii); and

v). a means of detecting said emitted light.

In a fourth aspect there is provided a multi-component detection system comprising:

i). a first agent comprising a first interacting group coupled directly or indirectly to a first tag, which first tag emits light of a first wavelength upon activation by a substrate or energy source which produces a first activated tag;

ii). a second agent comprising a second interacting group coupled directly or indirectly to a second tag, which second tag can accept the energy from the first tag in i) when the first and second interacting groups are associated and an appropriate substrate or energy source for the first tag in i) is present thereby producing a second activated tag that emits light of a second wavelength;

iii). a third agent comprising a third interacting group coupled directly or indirectly to a third tag consisting of a non-fluorescent quencher molecule that can accept the energy from:

a). the first activated tag when the first and third interacting groups are associated; and/or

b). the second activated tag when the second and third interacting groups are associated; and an appropriate substrate or energy source for the first and/or second tag is present, whereby the light emission from the first and/or second activated tag is decreased;

iv). an appropriate substrate or energy source to activate the tags in i) and ii); and

v). a means of detecting said emitted light.

In a fifth aspect there is provided a multi-component detection system comprising:

i). a first agent comprising a first interacting group coupled directly or indirectly to a first tag, which first tag emits light of a first wavelength upon activation by a substrate or energy source which produces a first activated tag;

ii). a second agent comprising a second interacting group coupled directly or indirectly to a second tag, which second tag emits light of a second wavelength upon activation by a substrate or energy source, which produces a second activated tag;

iii). a third agent comprising a third interacting group coupled directly or indirectly to a third tag, which third tag can accept the energy from the first activated tag when the first and third interacting groups are associated and an appropriate substrate or energy source for the first tag is present to produce a third activated tag that emits light of a third wavelength;

iv). a fourth agent comprising a fourth interacting group coupled directly or indirectly to a fourth tag, which fourth tag can accept the energy from the second activated tag when the second and fourth interacting groups are associated and an appropriate substrate or energy source for the second tag is present to produce a fourth activated tag that emits light of a fourth wavelength;

v). an appropriate substrate or energy source to activate the first and second tags, and

vi). a means of detecting said emitted light.

In a sixth aspect there is provided a multi-component detection system comprising:

i). a first agent comprising a first interacting group coupled directly or indirectly to a first tag, which first tag emits light of a first wavelength upon activation by a substrate or energy source, which produces a first activated tag;

ii). a second agent comprising a second interacting group coupled directly or indirectly to a second tag, which second tag can accept the energy from the first tag when the first and second interacting groups are associated and an appropriate substrate or energy source for the first tag is present thereby producing a second activated tag that emits light of a second wavelength;

iii). one or more further agents comprising one or more further interacting groups coupled directly or indirectly to one or more further tags that can accept the energy from the first activated tag when the first and one or more further interacting groups are associated and an appropriate substrate or energy source for the first tag is present to produce one or more further activated tags that emit light of one or more further wavelengths, wherein said further wavelengths are different to the first or second wavelengths;

iv). an appropriate substrate or energy source to activate the first tag, and

v). a means of detecting said emitted light.

In one embodiment, the interacting groups are capable of associating with one or more other interacting groups. These associations may be between identical interacting groups or between different interacting groups or combinations thereof.

Preferably, the interacting groups are selected from the group consisting of compounds, proteins, protein domains, protein loops, protein termini, peptides, hormones, lipids, carbohydrates, nucleic acids, oligonucleotides, pharmaceutical agents, pharmaceutical drug targets, antibodies, antigenic substances, viruses, bacteria, and cells or any associate or complex thereof.

When the interacting group is a nucleic acid molecule then any form of nucleic acid molecule may be used. For example, the nucleic acid molecule might include genomic deoxynucleic acid (DNA), recombinant DNA, complimentary DNA (cDNA), peptide nucleic acid (PNA), ribonucleic acid (RNA), RNA including hetero-nuclear RNA (hnRNA), transfer RNA (tRNA), small interfering RNA (siRNA), messenger RNA (mRNA), or ribosomal RNA (rRNA) and hybrid molecules thereof.

In one embodiment, external stimuli are applied to directly or indirectly modulate associations and/or conformations of interacting groups. Preferably, stimuli are reagents including any known molecule, organic or inorganic, proteinaceous or non-proteinaceous, ligand, antibody, enzyme, nucleic acid, carbohydrate, lipid, drug compound, agonist, antagonist, inverse agonist or compound or complex thereof or a change of conditions including temperature, ionic strength or pH.

Tags according to this invention may be any known molecule, organic or inorganic, proteinaceous or non-proteinaceous or complex thereof, capable of emitting energy including light or absorbing light in the near UV to near infra-red range or capable of fluorescence or phosphorescence. Preferably, the tag is a bioluminescent protein, a fluorescent protein, a fluorescent moiety or a non-fluorescent quencher.

Preferably, the bioluminescent protein is selected from the group consisting of luciferase, galactosidase, lactamase, peroxidase or any protein capable of luminescence in the presence of a suitable substrate.

Preferably, the fluorescent protein selected from the group consisting of green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz, GFPuv, destabilised EGFP (dEGFP), destabilised ECFP (dECFP), destabilised EYFP (dEYFP), HcRed, t-HcRed, DsRed, DsRed2, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin or any other protein capable of fluorescence or phosphorescence.

The fluorescent moiety can be any known fluorescent moiety. Preferably, the fluorescent moiety is selected from the group consisting of Alexa Fluor dyes and derivatives, Bodipy dyes and derivatives, Cy dyes and derivatives, fluorescein and derivatives, dansyl, umbelliferone, fluorescent and luminescent microspheres, fluorescent nanocrystals, Marina Blue, Cascade Blue, Cascade Yellow, Pacific Blue, Oregon Green and derivatives, Tetramethylrhodamine and derivatives, Rhodamine and derivatives, Texas Red and derivatives, rare earth element chelates or any combination or derivative thereof or any other molecule with fluorescent properties.

In one embodiment, at least one of the tags is a non-fluorescent quencher. The non-fluorescent quencher can be any known non-fluorescent chromophore with the ability to absorb light and to quench fluorescence and/or luminescence. The non-fluorescent quencher can therefore be any known proteinaceous or non-proteinaceous molecule. Preferably, the non-fluorescent quencher is selected from the group consisting of dabcyl, non-fluorescent pocilloporins, QSY-7, QSY-9, QSY-21, QSY-35, BHQ-1, BHQ-2 and BHQ-3.

The tags and interacting groups are directly or indirectly coupled. Preferably, the direct or indirect coupling is any known covalent or non-covalent means of coupling two molecules. More preferably, the direct or indirect coupling of the interacting groups and tags is selected from the group consisting of chemical cross-linking, chemical modification of proteins, chemical modification of amino acids, chemical modification of nucleic acids, chemical modification of carbohydrates, chemical modification of lipids or any other organic or inorganic molecule, non-covalent interactions including biotin-avidin, antigen-antibody or nucleic acid hybridisation.

In one preferred embodiment, the interacting group and tag are part of the same polypeptide chain. For example, a nucleic acid molecule coding for a proteinaceous interacting group and a proteinaceous tag are optionally fused to:

(i) a sequence coding for a peptide sequence used for affinity purification of a fusion construct; and/or

(ii) a sequence coding for a peptide sequence which directs the fusion construct to a subcellular compartment of a eukaryotic cell; and/or

(iii) a sequence coding for a peptide sequence which facilitates the penetration of a eukaryotic cell membrane to produce a fusion protein of the interacting group, tag and said peptide(s).

### BRIEF DESCRIPTION OF THE FIGURES

FIG. 1 shows the principle underlying a multiplex interaction assay.

FIG. 2 shows a simplified detection system for complex molecular associates. A signal from DT3 is only detected if DT1 and DT2 are both included in the associate.

FIG. 3 shows the principle of a detection system for complex molecular associates. DT1 is an energy donor for both DT2 and DT3 while DT2 is also an energy donor for DT3. Sequential excitation of DT1 and DT2 while detecting the emission from DT2 and DT3 or DT3, respectively yields information on the dynamic composition of the associate.

FIG. 4 shows fusion protein constructs. Schematic representation of the multiple cloning sites of pETDuet-1 (Novagen). PCR products were cloned in-frame into 4 different sites. Oligonucleotide linkers encoding for a 12- or 18-aminoacid spacer could be inserted between subunits 1 and 2. The open reading frame encoded by the multiple cloning site of the vector provided a 15-aminoacid spacer between subunits 1 and 3 and a 7-aminoacid spacer between subunits 2 and 3.

FIG. 5 shows spectral properties of proteinaceous DTs. Fluorescence spectra of ECFP, EGFP and mRFP1 (a) showed a large spectral overlap between ECFP and EGFP and some overlap between ECFP and mRFP1. There was significant spectral overlap between ECFP and EYFP and also EYFP and mRFP1 (b). The ECFP emission overlapped surprisingly well with the t-dimer2(12) excitation (c).

FIG. 6 shows FRET between proteinaceous DTs. The EYFP-12-ECFP (a) and t-dimer2(12)-12-ECFP (b) fusion proteins emitted additional light due to resonance energy transfer when excited at 440 nm. Spectra were normalised to the donor emission maximum and emission from direct excitation of the acceptor fluorophores by the light source was subtracted.

FIG. 7 shows RET between Renilla luciferase (Rluc), a bioluminescent protein and proteinaceous DTs: (a) no DT; (b) EGFP; (c) EYFP; (d) t-dimer2(12) and (e) mRFP1. Spectra were normalised to the emission maxima.

FIG. 8 shows RET ratios for various fusion proteins. Shown are the ratios for the EGFP and EYFP channels (a) and the t-dimer2(12) and mRFP1 channels (b). Good separation was achieved between EGFP-t-dimer2(12) and EYFP-t-dimer2(12), whereas EGFP-EYFP and t-dimer2(12)-mRFP1 are too close for an independent, simultaneous detection. Although mRFP1 was separated well from EGFP and EYFP it was not substantially activated by Rluc resulting in only a weak RET signal.

FIG. 9 shows an analysis of RET with non-proteinaceous DTs. Biotinylated Rluc was mixed with various streptavidin conjugates. Luminescence spectra are shown in black, fluorescence emission and excitation spectra of the conjugated dyes are shown in grey solid and dashed lines, respectively. The following conjugates were used: (a) Alexa Fluor 488, (b) Oregon green, (c) Alexa Fluor 555, (d) Alexa Fluor 568 and (e) Alexa Fluor 594. As a negative control non-biotinylated Rluc was used (f) which did not result in a RET signal.

FIG. 10 shows RET ratios depending on the concentration of the non-proteinaceous DTs. Solutions containing biotinylated Rluc mixed with varying amounts of either streptavidin-Oregon green or streptavidin-Alexa Fluor 594 were analysed. The concentrations ranged from equimolar amounts of streptavidin and biotin-Rluc to biotin-Rluc without a streptavidin conjugate. For both conjugates the RET ratio was found to be above the background ratio even at the lowest concentration.

FIG. 11 shows in vitro multiplex RET detection. Mixtures of EGFP-15-Rluc/t-dimer2(12)-15-Rluc (a) and streptavidin-Oregon green/streptavidin-Alexa Fluor 594 (b) were analysed. In both models the 2 channels could be analysed simultaneously and quantitated independently. The RET ratios of the two labels were indicative of the extent by which the first (donor) DT interacts with the second and/or third DTs.

FIG. 12 shows spectral FRET detection in a cell-based assay to determine the association of G-protein coupled receptors (GPCRs) with each other. The homodimerisation between CCR2 receptors (a) and TRH receptors (b) was monitored using receptors that were C-terminally fused to ECFP, EYFP or t-dimer2(12). Both receptors formed homodimers as was detected by an increase of the EYFP or t-dimer2(12) emissions due to RET. Both signals were detected simultaneously in the presence of all three fusion constructs representing combinations of dimmers or the formation of larger oligomeric complexes (b). Spectra were normalised to the ECFP emission maximum at 480 nm and emission from direct excitation of the acceptor fluorophores by the light source was subtracted.

FIG. 13 shows a numerical analysis of FRET between GPCRs in homodimer complexes in live mammalian cells. The peak areas of the EYFP and t-dimer2(12) were calculated by integration of the fluorescence emission spectra (FIG. 12). The homodimer complexes were detected for both CCR2 (a) and TRHR (b) using either of the acceptor DTs. The absolute signals obtained in the presence of all three fusion constructs (b) were lower, albeit still above background, due to the transient transfection system resulting in lower co-transfection efficiencies. Although the interaction between CCR2 receptors was weaker or in a less favourable conformation it was still easily detected.

FIG. 14 shows the spectral analysis of a cell based, multiplex assay for the detection of the ligand induced interaction between different GPCRs with beta-arrestin-2, a downstream effector protein. Beta-arrestin-2, N-terminally fused to Rluc, interacts with both TRHR and CCR2 after addition of an appropriate ligand. TRHR was C-terminally fused to EYFP and CCR2 to t-dimer2(12) (a) or vice versa (b). Addition of TRH, a ligand specific for TRHR resulted in RET specific for the TRHR:beta-arrestin-2 interaction. Addition of MCP1, a ligand specific for CCR2, activated this receptor as was observed in an increase of the respective RET signal with beta-arrestin-2. Adding both ligands activated both receptors and resulted in both receptors interacting with beta-arrestin-2 and thus, two RET signals were detected.

FIG. 15 shows a numerical analysis of a cell based, multiplex assay for the detection of the ligand induced interaction between different GPCRs with beta-arrestin-2, a downstream effector protein. Emission spectra from FIG. 14 were integrated to determine the peak areas of EYFP and t-dimer2(12) peaks. Depending on the presence of the ligands, beta-arrestin-2:TRHR and beta-arrestin-2:CCR2 were detected independently, using C-terminal fusions of TRHR-EYFP and CCR2-t-dimer2(12) (a) or vice versa (b).

FIG. 16 shows a simplified detection system for complex molecular associates exemplified by fusion proteins consisting of Rluc, EGFP and mRFP1. RET was observed between EGFP and mRFP1 when the fusion protein was excited at 480 nm (a). Energy transfer from Rluc to mRFP1 was higher in the presence of EGFP as was indicated by a higher emission between 600-650 nm (b).

FIG. 17 shows a detection system for complex molecular associates exemplified by fusions of fluorescent proteins. ECFP was able to activate both EYFP and mRFP1 (a), and EYFP also activated mRFP1 (b).

FIG. 18 shows the detection system using a combination of proteinaceous and non-proteinaceous, small molecule dyes. The excitation spectra of Alexa Fluor 555 and Alexa Fluor 568 overlapped well the emission spectra of ECFP and EYFP (a). In this model system, DT1 and DT2 were present as a biotinylated fusion protein of ECFP and EYFP which interacts with streptavidin conjugated to the dye. Alexa Fluor 555 was activated by both ECFP (b) and EYFP (c). As a control the biotin streptavidin-conjugate interaction was blocked by preincubation using an excess of unconjugated streptavidin, which significantly reduced the Alexa Fluor emission. The same effects were observed using an Alexa Fluor 568 conjugate, which was activated by ECFP (d) and EYFP (e) resulting in a weaker signal but better spectral resolution compared to the more blue-shifted Alexa Fluor 555. Due to the greater spectral resolution the Alexa Fluor 568 emission due to direct excitation by the light source was also reduced in the control containing unconjugated streptavidin.

FIG. 19 shows a numerical analysis of the FRET between ECFP, EYFP and Alexa Fluor 555 (a) or Alexa Fluor 568 (b) by spectral peak integration and calculation of RET ratios. A signal above background was observed for the ECFP-EYFP interaction as well as the biotin-streptavidin interaction. The presence of the Alexa Fluor and EYFP signals at 440 nm excitation as well as the presence of the Alexa Fluor signals at 490 nm excitation indicated that a complex containing all DTs was formed.

FIG. 20 shows a detection system for complex molecular associates exemplified by tagged receptors present in the cell membrane of live mammalian cells. RET from ECFP to EYFP and from ECFP to ECFP was observed when ECFP was excited at 440 nm, indicating the homodimer formation between the CCR2 receptors (a). RET was also observed between EYFP and mRFP1 when EYFP was excited at 490 nm additionally indicating the association of CCR2-EYFP and CCR2-mRFP1. A numerical analysis of the peak areas indicated signal increases accurately reflecting molecular associations (c).

### ABBREVIATIONS

BRET Bioluminescence resonance energy transfer
CCR2 Chemokine (CC motif) receptor 2.
DT Tag or detection tag.
DT-IG Tag or detection tag attached to an interacting group.
ECFP Enhanced Cyan Fluorescent Protein, which is a variant of the Aequorea victoria green fluorescent protein gene (GFP).
EGFP Enhanced Green Fluorescent Protein is a red-shifted variant of wild-type GFP.
EYFP Enhanced Yellow Fluorescent Protein.
FRET Fluorescence resonance energy transfer.
GPCRs G-protein coupled receptors.
His(6) Histidine tag consisting of 6 consecutive histidine residues.
IG Interacting group.
mRFP1 Monomeric red fluorescent protein.
RET Resonance energy transfer.
Rluc Renilla luciferase.
T7 prom. T7 promoter sequence
T7 stop T7 terminator sequence.

### DETAILED DESCRIPTION OF THE INVENTION

Before describing the present invention in detail, it is to be understood that this invention is not limited to particularly exemplified bioluminescent or fluorescent proteins, analytes, or methods disclosed herein, which may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols, reagents and vectors which are reported in the publications and which might be used in connection with the invention. Nothing herein is to be construed as an admission that the invention is not entitled to antedate such disclosure by virtue of prior invention.

Furthermore, the practice of the present invention employs, unless otherwise indicated, conventional molecular biology, chemistry and fluorescence techniques, within the skill of the art. Such techniques are well known to the skilled worker, and are explained fully in the literature. See, eg., Coligan, Dunn, Ploegh, Speicher and Wingfield “Current protocols in Protein Science” (1999) Volume I and II (John Wiley & Sons Inc.); and Bailey, J. E. and Ollis, D. F., Biochemical Engineering Fundamentals, McGraw-Hill Book Company, NY, 1986; Lakowicz, J. R. Principles of Fluorescence Spectroscopy, New York: Plenum Press (1983) for fluorescence techniques.

It must be noted that as used herein and in the appended claims, the singular forms “a,” “an,” and “the” include plural reference unless the context clearly dictates otherwise. Thus, for example, a reference to “a protein” includes a plurality of such proteins, and a reference to “an analyte” is a reference to one or more analytes, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The present invention relates to a system for detecting multiple molecular associations. The term “molecular association” or “association” as used herein refers to a combination of two or more interacting groups associated via any known direct or indirect stabilising atomic or molecular level interaction or any combination thereof, where the interactions include, without limitation, bonding interactions such as covalent bonding, ionic bonding, hydrogen bonding, co-ordinate bonding, or any other molecular bonding interaction, electrostatic interactions, a polar or hydrophobic interactions, or any other classical or quantum mechanical stabilising atomic or molecular interaction.

In one embodiment, the molecular association is between one or more agents comprising one or more interacting groups (IGs), wherein the IGs are coupled directly or indirectly to one or more tags (“DTs”). The term “agent” or “IG-DT agent” as used herein refers to a complex between an IG and a tag (“DT”), i.e. an IG coupled directly or indirectly to a DT. Agents may be engineered or modified to contain chemical groups, peptide sequences, proteins or nucleic acid molecules that may (i) facilitate their purification and/or (ii) target them to a subcellular compartment of a eukaryotic host cell and/or (iii) enable them to penetrate the cell membrane of a eukaryotic cell when added to the medium surrounding the cell.

In one embodiment, the agents may be a plurality of agents in that the detection system is capable of discriminating the association of any number of molecules. However, in a further embodiment, the detection system of the invention consists essentially of a first, second and third agent.

Accordingly, the term “association” also refers to any interaction or conformational change involving interacting groups that brings the coupled tags into proximity. The distance between the tags is preferably in the range of between 1 and 10 nm. A direct physical contact between the IG-DT agents is not required and may be mediated by one or more additional molecule(s) and/or one or more additional interacting group(s).

The term “interacting group” or “IG” as used herein encompasses compounds, proteins, protein domains, protein loops, protein-termini, peptides, hormones, protein-lipid complexes, lipids, carbohydrates, carbohydrate-containing compounds, nucleic acids, oligonucleotides, pharmaceutical agents, pharmaceutical drug targets, antibodies, antigenic substances, viruses, bacteria, and cells or any complex thereof. Essentially, the interacting group is an entity capable of forming a complex with one or more entities. For example, an antibody in context with the present invention would be a first IG in that it is capable of forming a complex with an antigen, wherein the antigen would be the second IG (see infra). Another example of an IG of the present invention would be a ligand, which is capable of forming a complex with a receptor. A further example is the interaction of an enzyme with its substrate. Additionally, the IGs may be part of the same molecule. Accordingly, for example, the third intracellular loop of a G-protein coupled receptor could be a first IG and the C-terminus of the same receptor could be a second IG which would associate when the receptor is activated or inactivated.

In one embodiment, external stimuli are applied to directly or indirectly modulate associations and/or conformations of interacting groups. The term “stimuli” as used herein refers to reagents including any known molecule, organic or inorganic, proteinaceous or non-proteinaceous, ligand, antibody, enzyme, drug compound, agonist, antagonist, inverse agonist, compound or complex thereof. It further refers to a change of external conditions including temperature, ionic strength or pH. Stimuli can act directly or indirectly. For example if stimuli are reagents they may physically bind to interacting groups and consequently mediate or prevent their association. This for example, could be a ligand that results in the dimerisation of a receptor or a conformational change within a receptor. An example for stimuli acting indirectly would be a reagent or change of conditions that activates an intracellular signalling pathway with the result that IGs are modified by cellular enzymes, for example phosphorylated; the modification in turn changes the associations of the IGs.

The term “tag” as used herein encompasses bioluminescent proteins, fluorescent proteins, fluorescent moieties and non-fluorescent quenchers. In short any known molecule, organic or inorganic, proteinaceous or non-proteinaceous or complex thereof, capable of emitting energy such as light or absorbing light in the near UV to near infra-red range or capable of fluorescence or phosphorescence.

The term “bioluminescent protein” as used herein refers to any protein capable of generating luminescence. Bioluminescent proteins include luciferases, which have been found in bacteria, fungi, insects and marine creatures. They catalyse the oxidation of a specific substrate (generally known as luciferins) under light emission (Hastings (1996) Gene 173, 5-11). The most widely known substrate is coelenterazine which occurs in cnidarians, copepods, chaetgnaths, ctenophores, decapod shrimps, mysid shrimps, radiolarians and some fish taxa (Greer & Szalay, (2002), Luminescence, 17, 43-74). Two of the most widely used luciferases are:

(i) Renilla luciferase (from R. reniformis), a 35 kDa protein, which uses coelenterazine as a substrate and emits light at 480 nm (Lorenz et al., (1991), PNAS. USA, 88, 4438-4442); and

(ii) Firefly luciferase (from Photinus pyralis), a 61 kDa protein, which uses luciferin as a substrate and emits light at 560 nm (de Wet et al., (1987), Mol. Cell. Biol., 2987, 725-737).

More recently, Gaussia luciferase (from Gaussia princeps) has been used in biochemical assays (Verhaegen et al., (2002), Anal. Chem., 74: 4378-4385). Gaussia luciferase is a 20 kDa protein that oxidises coelenterazine in a rapid reaction resulting in a bright light emission at 470 nm.

In one embodiment, the bioluminescent proteins used with the present invention exhibit an intense and constant light emission as long as the substrate is present. As the bioluminescent proteins are coupled to IGs, it is preferable to use bioluminescent proteins with a small molecular weight to prevent an inhibition of the interaction between the IGs due to steric hindrance. The bioluminescent proteins preferably consist of a single polypeptide chain to facilitate an easy production of the IG-DT agent. Also the bioluminescent proteins preferably do not form oligomers or aggregates, which could otherwise inhibit the function of the coupled IG. The bioluminescent proteins Renilla luciferase, Gaussia luciferase and Firefly luciferase meet all or most of these criteria.

The term “substrate” as used herein refers to any molecule that can be used in conjunction with a bioluminescent protein to generate or absorb luminescence.

The choice of the substrate can impact on the wavelength and the intensity of the light generated by the bioluminescent protein. For Renilla luciferase for example, coelenterazine analogues are available that result in light emission between 418 and 512 nm (Inouye et al., (1997), Biochem. J., 233, 349-353). A coelenterazine analogue (400A, ‘DeepBlueC’) has been described emitting light at 400 nm with Renilla luciferase (PCT application WO01/46691).

Substrates used with this invention are preferably cell-permeable and are able to pass the cellular membrane to become available to an intracellular bioluminescent protein. Coelenterazine and most of its derivatives are highly cell permeable (Shimomura et al., (1997), Biochem. J., 326: 297-298), whereas luciferin does not efficiently cross the membrane of mammalian cells. However, a caged luciferin compound has been developed that passes the cell membrane and is released by cellular enzymes or UV light once inside the cytoplasm (Yang et al., (1993), Biotechniques, 15, 848-850.

The term “fluorescent protein” as used herein refers to any protein capable of fluorescence or phosphorescence. There are a number of different fluorescent proteins that can be employed in this invention. For example, the most widely used fluorescent protein in molecular and cell biology are the green fluorescent protein (GFP) from the jellyfish Aequorea victoria (Tsien, (1998), Annu. Rev. Biochem., 67, 509-544) and the variants derived from its sequence. ‘Enhanced’ fluorescent proteins (e.g. EGFP) were developed by point mutations that increase the solubility and fluorescence and accelerate protein folding (Zernicka-Goetz et al., (1997), Development, 124, 1133-1137). A Phe to Leu point mutation at position 64 has increased stability of the protein at 37° C. and a Ser to Thr mutation at position 65 resulting in an increased fluorescence (Okabe et al., (1997), FEBS Letters, 407, 313-319; Clontech Palo Alto, Calif.). The EGFP which is commercially available from Clontech incorporates a humanised codon usage rendering it “less foreign” to mammalian transcriptional machinery and ensuring maximal gene expression. Additionally, the spectral properties of the green fluorescent protein can be altered by site-directed mutagenesis of specific amino acids, for example blue (EBFP), cyan (ECFP) and yellow (EYFP) mutants of EGFP have been produced (Zhang et al., (2002), Nat. Rev. Mol. Cell Biol., 3, 906-918). Another important class of fluorescent proteins is the red fluorescent proteins (RFP) from the coral species Discosoma (DsRed) (Matz et al., (1999), Nature Biotechnol. 17, 969-973) and Heteractis crispa (HcRed) (Gurskaya et al., (2001), FEBS Lett. 507, 16-20).

Preferably fluorescent proteins with a high fluorescence quantum yield are used with the present invention.

Preferably, the molecular weight of fluorescent proteins used with the present invention should be small enough to avoid steric hindrance between the IGs.

Preferably, monomeric proteins are used to avoid aggregation and interference with the function of a coupled IG. GFP forms a weak dimer but its tendency to dimerise can be minimised by the mutation of hydrophobic amino acids in the dimerisation interface (Zacharias et al., (2002), Science, 296, 913-916). The red fluorescent protein DsRed is an obligate tetrameric protein. Recently, 17 point mutations of the DsRed sequence have been described that render DsRed into a dimeric protein (dimer2). The subunits of the dimer can be connected via a peptide linker to form a tethered dimer (t-dimer2(12)) that physically acts as a monomer. Additional 16 point mutations convert the dimer2 into a monomeric variant (mRFP1) (Campbell et al., (2002), PNAS. USA, 99, 7877-7882). The red fluorescent protein HcRed is a dimeric protein and is not fluorescent as a monomer. However, the two subunits can be fused by a short peptide linker connecting the C-terminus of the first subunit with the N-terminus of the second. This fusion protein (t-HcRed) acts effectively as a monomeric unit, similar to t-dimer2(12) (Fradkov et al., (2002), Biochem. J., 368, 17-21).

Preferably, fluorescent proteins used with the present invention exhibit short maturation times for the formation of their fluorophores. The fluorophore in these molecules is formed by specific re-arrangements of the polypeptide chain. This process can take from less than 1 h to more than 24 h (Zhang et al., (2002), Nat. Rev. Mol. Cell Biol., 3, 906-918). As a slow maturation process limits the availability and concentration of functional DT, the use of rapidly maturing proteins is preferred. Rapidly maturing fluorescent proteins are for example the green fluorescent protein EGFP and its colour variants and the red fluorescent proteins t-dimer2 and mRFP1. Slow maturing proteins are for example DsRed and HcRed.

The terms “fluorescent moiety” or “fluorescent moieties” are used herein interchangeably and refer to non-proteinaceous molecules that are capable of generating fluorescence. Non-proteinaceous fluorescent molecules are usually small molecules that can be attached to other molecules. Each non-proteinaceous fluorescent molecule has specific spectral characteristics. There are a number of different fluorescent moieties that can be employed in this invention. Non-limiting examples include rhodamine, rhodamine derivatives, dansyl, umbelliferone, fluorescein, fluorescein derivatives, Oregon green, Texas Red, Alexa Fluor dyes and Cy dyes. A very attractive class of fluorescent moiety with regards to this invention are fluorescent nanocrystals (Bruchez et al., (1998), Science, 281, 2013-2016). Fluorescent nanocrystals exhibit a strong fluorescence and their fluorescence emission can be adjusted by the crystal size over a wavelength range of more than 1000 nm. The excitation of all nanocrystals occurs at the same wavelength independent of their fluorescence emission. Therefore, various nanocrystals can be excited by the same light source or via RET from the same bioluminescent protein or fluorescent molecule.

Preferably fluorescent moieties with high fluorescence quantum yields are used.

A new type of fluorescent moiety was reported recently and involves both proteinaceous and non-proteinaceous components (Griffin et al., (1998), Science, 281, 269-272; Adams et al., (2002), J. Am. Chem. Soc., 124, 6063-6076). The biarsenical-tetracysteine system fuses a short tetracysteine containing peptide to a target protein. This peptide forms a stable, fluorescent complex with a cell-permeable, non-fluorigenic biarsenical dye. Depending on the molecular structure of the dye different fluorophores are obtained.

The term “energy source” as used herein refers to any energy source capable of activating a specific fluorophore. In one preferred embodiment, the energy source is light. Non-limiting examples of light sources include lasers, Hg-lamps or Xe-lamps. The light source further has a means of limiting the emitted light to a specific wavelength or a specific range of wavelengths. This can be, for example, a suitable filter mounted to a filter wheel or a filter slide, a monochromator or lasers that only produce light of a single wavelength.

The term “non-fluorescent quencher” refers to any known proteinaceous or non-proteinaceous molecule, which is capable of absorbing fluorescence light without emitting light itself. Non-limiting examples are dabcyl, QSY quenchers, BHQ quenchers and non-fluorescent pocilloporin pigment proteins.

With regards to this invention, the bioluminescent, fluorescent proteins or fluorescent moieties should have suitable spectral properties for resonance energy transfer (RET) as well as certain physical characteristics. Their light emission should preferably be intense and constant as long as the necessary substrate is present. As the bioluminescent proteins and/or fluorescent moieties can be coupled directly or indirectly to IGs, it is most desirable to use small bioluminescent and fluorescent proteins to prevent an inhibition of the interaction between the IGs due to steric hindrance.

The terms “coupled directly or indirectly” as used herein means that the tag is attached to or associated with the IG to form an agent which is capable of being analysed or detected. The preferred method of coupling is determined by the nature of the IGs and DTs.

The bioluminescent or fluorescent proteins may be coupled (e.g., covalently bonded) to a suitable IG either directly or indirectly (e.g., via a linker group). Means of coupling bioluminescent or fluorescent protein to an agent are well known in the art. An example of a direct method of coupling a proteinaceous IG and a proteinaceous DT is genetic fusion, wherein the genes encoding the IG and the bioluminescent or fluorescent protein are fused to produce a single polypeptide chain.

Another example of a direct coupling method is conjugation, wherein the coupling of the IG with the fluorophore uses enzymes such as ligases, hydrolases, particularly phosphatases, esterases and glycosidases, or oxidoreductases, particularly peroxidases.

Fluorescent moieties and non-proteinaceous, non-fluorescent quenchers have the disadvantage that their attachment to proteinaceous IGs is more difficult and often cannot occur inside live cells, in contrast to proteinaceous fluorescent moieties that can be genetically fused to proteinaceous IGs. An example of direct coupling of non-proteinaceous fluorescent moieties and non-fluorescent quenchers to IGs involves moieties covalently linked to reactive groups, which are able to form a covalent bond with specific chemical groups of the IG. Examples are iodoacetamides and maleimides reacting with SH-groups of cysteine residues, and succinimidyl esters, carboxylic acids and sulfonyl chlorides reacting with NH³⁺-groups of lysine residues (Ishii et al., (1986), Biophys. J. 50, 75-89; Staros et al., (1986), Anal. Biochem. 156, 220-222; Lefevre et al., (1996), Bioconjug. Chem. 7, 482-489).

Another known way to attach a fluorescent moiety or a non-fluorescent quencher to the IG typically involves grafting a fluorescent moiety onto the IG or by incorporating the fluorescent moiety into the IG during its synthesis. It is important that the labelled IG retains the critical properties of the unlabelled IG such as selective binding to a receptor or nucleic acid, activation or inhibition of a particular enzyme, or ability to incorporate into a biological membrane. There are a wide variety of fluorescent moieties available, including for example, dipyrrometheneboron difluoride dyes, rhodamine, rhodamine derivatives, Texas Red, dansyl, umbelliferone, etc. For a review of various labelling or signal producing systems that may be used, see U.S. Pat. No. 4,391,904.

One example of an indirect method of coupling a fluorescent moiety or non-fluorescent quencher to an IG such as a protein or nucleic acid, involves the covalent bonding of the fluorescent moiety or non-fluorescent quencher to a protein such as avidin, which is capable of binding biotin, wherein the biotin is covalently bound to the IG such that the IG and the fluorescent moiety or non-fluorescent quencher are coupled indirectly together via the interaction between biotin and avidin.

Another example of an indirect method of coupling the IG and bioluminescent or fluorescent protein is via a linker group. A linker group can function as a spacer to distance the bioluminescent or fluorescent protein from the agent in order to avoid interference with binding capabilities. A linker group can also serve to increase the chemical reactivity of a substituent on an agent, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalogue of the Pierce Chemical Co., Rockford, Ill.), may be employed as the linker group. Coupling may be effected, for example, through amino groups, carboxyl groups, sulfhydryl groups or oxidised carbohydrate residues. There are numerous references describing such methodology, e.g., U.S. Pat. No. 4,671,958.

In one embodiment a proteinaceous DT or a proteinaceous IG is produced recombinantly by inserting a DNA sequence that encodes a DT or IG into an expression vector by standard molecular biology techniques well known to those skilled in the art. The DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide. The polypeptide of the fused DT and IG is expressed in an appropriate host. Any of a variety of expression vectors known to those of ordinary skill in the art may be employed to express recombinant polypeptides of this invention. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast or a mammalian cell line, such as CHO cells.

In another embodiment a proteinaceous IG-DT agent is produced recombinantly as a fusion construct. A DNA sequence encoding a fusion protein of the present invention is constructed using known recombinant DNA techniques to assemble separate DNA sequences encoding the proteinaceous DT polypeptide and the IG polypeptide into an appropriate expression vector. The 3′ end of the first DNA sequence is ligated, with or without a peptide linker, to the 5′ end of the second DNA sequence so that the reading frames of both sequences are in phase to permit mRNA translation of the two DNA sequences into a single fusion protein that retains the biological activity of both the DT and IG. The orientation of DT and the IG within the fusion construct may be swapped to increase its functionality or expression.

A peptide linker sequence may be employed to separate the bioluminescent protein and IG polypeptide by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the bioluminescent protein or IG; and (3) the lack of hydrophobic or charged residues that might react with the polypeptide functional epitopes or decrease the solubility of the fusion protein. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., (1985), Gene, 40, 39-46; Murphy et al., (1986), PNAS. USA, 83, 8258-8262; U.S. Pat. Nos. 4,935,233 and 4,751,180. The linker sequence may be from 1 to about 50 amino acids in length. Peptide sequences are not required when the bioluminescent protein or IG have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons that are required to end translation and transcription termination signals are only present 3′ to the DNA sequence encoding the second polypeptide.

In one embodiment the sequence encoding the recombinant polypeptide is further genetically fused to a sequence encoding a peptide that facilitates the purification of the fusion construct via affinity chromatography. Examples include histidine tags, maltose-binding protein tags, cellulose-binding protein tags, intein tags, S-tags and GST tags.

In another embodiment the sequence encoding the recombinant polypeptide is genetically fused to a sequence encoding a peptide that facilitates the targeting of the fusion construct to a specific subcellular compartment of a eukaryotic host cell or for secretion into the surrounding medium. Examples include nuclear localisation signals, mitochondrial import sequences, KDEL sequences to target the endoplasmatic reticulum and export signals.

In yet another embodiment the sequence encoding the recombinant polypeptide is genetically fused to a sequence encoding a peptide that facilitates the penetration of eukaryotic cell membranes and thus the uptake of the fusion construct into the cell (Schwartz et al., (2000), Curr. Opin. Mol. Ther., 2, 162-167). Examples include peptide sequences derived from the HIV Tat protein, Herpes simplex virus VP22 and Kaposi FGF-4.

As an alternative to recombinant methods, polypeptides and oligopeptides can be chemically synthesised. Such methods typically include solid-state approaches, but can also utilise solution based chemistries and combinations or combinations of solid-state and solution approaches. Examples of solid-state methodologies for synthesising proteins are described by Merrifield, (1964), J. Am. Chem. Soc., 85, 2149; and Houghton, (1985), PNAS. USA., 82, 5132.

Once the IGs have been labelled with the tags as described above, they can then be reacted with one or more other IGs, which also have attached thereto one or more tags.

In one embodiment all IG-DT agents are proteinaceous and coupled by genetic fusion to express IG-DT fusion constructs in a suitable host cell. The activation and detection of the DTs as well as an association of the IGs occurs inside the living host cell, inside cellular organelles, inside its cell membrane or at its surface.

In another embodiment a subset of IG-DT agents is proteinaceous and coupled by genetic fusion to express IG-DT fusion constructs in a suitable host cell. Another subset of IG-DT agents, proteinaceous, non-proteinaceous or combinations thereof, is added to the host cell with the optional ability of penetrating the host cell membrane. The activation and detection of the DTs as well as an association of the IGs occurs inside the living host cell, inside cellular organelles, inside its cell membrane or at its surface.

In yet another embodiment the IG-DT agents, regardless of their nature and of the method of preparations, are provided in solutions that may also contain suitable buffer substances. The IG-DT agents may be part of a cell extract, a cell fraction or a synthesis mixture, or may be at least about 90% pure, more preferably at least about 95% pure and most preferably at least about 99% pure. Purification occurs according to standard procedures of the art, including ammonium sulphate precipitation, affinity columns, ion exchange and/or size exclusion and/or hydrophobic interaction chromatography, HPLC, FPLC, gel electrophoresis, capillary electrophoresis and the like (see, generally, Scopes, (1982), Protein Purification, Springer-Overflag, N.Y., Deutsche, Methods in Enzymology Vol. 182: Guide to Protein Purification, Academic Press, Inc. N.Y. (1990)).

The present invention involves combinations of pairs of DTs, capable of being a donor and/or acceptor molecule. Accordingly, the DTs that can be used according to the present invention can be selected based on the physical properties thereof, as is known in the art of resonance energy transfer (RET), the two being selected so that they together comprise the donor and acceptor molecules of a RET pair. If one of the DTs within a RET pair is a bioluminescent protein, the RET is known as bioluminescence RET (BRET). If both DTs forming a RET pair are fluorophores the resulting RET is known as fluorescence RET (FRET). Examples of known suitable donor and acceptor pairs include:

Renilla luciferase and yellow fluorescent protein;

Renilla luciferase and green fluorescent protein;

Cyan fluorescent protein and yellow fluorescent protein;

fluorescein and tetramethylrhodamine;

5-(2′-aminoethyl) aminonaphthalene-1-sulfonic acid (EDANS) and fluorescein;

See generally R. Haugland, Handbook of Fluorescent Probes and Research Chemicals (Sixth Ed. 1995). One or both of the fluorophores can be a fluorescent protein such as green fluorescent protein, and it is particularly advantageous to employ a fluorescent protein as the fluorophore when the test compound is a protein or peptide by preparing a fusion protein of the test compound and a fluorescent protein.

The present invention involves the detection of multiple RET signals in parallel and combinations of bioluminescent or fluorescent moieties with specific spectral characteristics must be chosen. General spectral requirements and examples for combinations of these bioluminescent or fluorescent moieties depending on different embodiments as well as examples of applications are described below:

(i) Multiplex Detection System—‘OR’ Assays

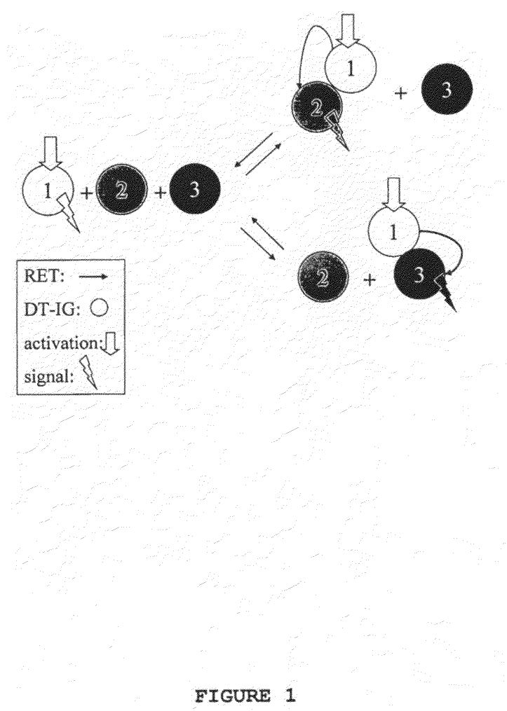

In one embodiment the emission spectrum of a first acceptor tag (DT1) sufficiently overlaps with the excitation spectra of both the second tag (DT2) and subsequent tags (DT3+), while the emission maxima of DT1, DT2 and DT3 are sufficiently distinct to allow their separate detection (see, for example, FIG. 1 and Table 1).

TABLE 1
TYPES OF ASSAYS AND EXPECTED SIGNALS UPON
ACTIVATION OF DT1 BY AN APPROPRIATE SUBSTRATE
OR EXCITATION LIGHT

| Assay type | IG association | DT1 activates* |
|---|---|---|
| multiplex | 1:2 | 2 |
| | 1:3 | 3 |
| simple detection | 1:2 | 2 |
| system for complex | 1:2:3 | (2) + 3 |
| molecular associates | | |

*Numbers indicate an increased signal of this DT.

An example of a suitable combination of DTs is Renilla luciferase (emitting at 460-490 nm) or ECFP to be used as DT1 in combination with EGFP or EYFP (DT2) and DsRed, dimer2 or t-dimer2(12) (DT3). Although DsRed and the dimeric variants absorb only weakly below 500 nm they form a surprisingly strong RET acceptor. Alternatively, the biarsenical dyes FlAsH and ReAsH may be used as DT2 and DT3 (Adams et al., (2002), J. Am. Chem. Soc., 124, 6063-6076). Another alternative for DT2 and DT3 are fluorescent moieties with similar spectral properties as EGFP or EYFP and DsRed. Examples include Alexa Fluor 488, Oregon green 514 and Alexa Fluor 546. Yet another alternative for DT2 and DT3 are fluorescent nanocrystals. All nanocrystals absorb light below 500 nm, independent of their emission wavelength (Bruchez et al. (1998), Science, 281, 2013-2016), making them ideal RET acceptors for this type of assay.

A variation of this embodiment allows the monitoring of two independent interactions involving IGs which are capable of pairwise interactions, i.e. IG1:IG2 and IG3:IG4. In this embodiment DT1 is directly or indirectly linked to IG1 and IG3 while DT2 may be linked to IG2 and DT3 to IG4. The spectral requirements of the DTs remain unchanged.

One example of an application of this embodiment of the invention is the monitoring of signal transduction pathways. Most cellular signalling events involve networks of interacting proteins, relaying a signal from a receptor to a response, usually involving gene transcription in the nucleus. The IGs can be components of a signalling pathway with IG1 relaying a signal to IG2 and IG3 or IG1 acting as the signalling link between IG2 and IG3. Examples of signalling molecules of which the IGs can be derived from are ras and raf proteins, protein kinase C, MEK proteins etc. (Dikic et al., (1999), Cell Biochem. Biophys., 30, 369-387; Gutkind et al., (1988), Oncogene, 17, 1331-1342; Luttrell et al., (1999), Curr. Opin. Cell Biol. 11, 177-183; Rozengurt et al., (1998), J. Cell Physiol. 177, 507-517).

Another example is the transcriptional regulation of gene expression. Transcription factors act in multiprotein-DNA complexes and the composition of these complexes determines their specificity and activity (Wolberger et al., (1999), Ann. Rev. Biophys. Biomol. Struct., 28, 29-56). For example, the transcription factor Fos forms hetero-dimers with different members of the Jun transcription factor family, depending on the cellular differentiation, growth, external stimuli etc. (Chinenov et al. (2001), Oncogene, 20, 2438-2452). IGs can be derived from Fos and Jun family members monitoring selectively the state and activity of these important transcriptional regulators.

A further example of the application of this embodiment of the invention is the monitoring of two different parts of a cellular signal transduction cascade. Signals are relayed from the activated receptor to their effective intracellular site via a cascade of interacting and each other activating or deactivating proteins. A well-characterised example is the MAPK/Erk pathway (Cobb et al. (1999), Prog. Biophys. Mol. Biol., 71: 479-500; Lewis et al., (1998), Adv. Cancer Res. 74, 49-139). The MAPK/Erk signalling cascade is activated by a wide variety of receptors involved in growth and differentiation including receptor tyrosine kinases (RTKs), integrins, and ion channels. Pairs of IGs may be derived from different interacting pairs of signalling molecules of the cascade. Each interacting pair gives a specific RET signal (DT1-IG1:IG2-DT2 and DT1-IG3:IG4-DT3) indicating the activation of a specific step. This may allow the simultaneous monitoring of one step upstream in the cascade (e.g. SOS-Ras) and another one further downstream (e.g. MEK-Erk). This type of assay is useful to identify components of the cascade that lie between the two detected steps. In high-throughput screening and drug discovery it may be used for the identification of drugs manipulating molecules between the two detected steps.

In another example, the assay is used to distinguish between the function of a mutant versus a normal protein. The role of mutant activated receptor protein-tyrosine kinases (PTKs) in oncogenesis is well established. An important principle in the activation of receptor PTKs is ligand-mediated dimerisation. Increasing evidence indicates that oncogenic activation of receptor PTKs occurs through mutations that lead to constitutive dimerisation and activation of the cytoplasmic catalytic domain (Hunter et al. (1997), Cell, 88, 333-346). One example is the Tel-PDGFb receptor fusion, generated by the t(5:12) translocation in chronic myelomonocytic leukaemia. The N-terminal part of Tel, an Ets family transcription factor, is joined with the entire cytoplasmic domain of the PDGFb receptor PTK gene, resulting in dimerisation and constitutive PTK activation (Golub et al., (1994), Cell, 77, 307-316). The assay system provided in this invention derives IGs from the receptor components and monitors the activity of both the defective (mutant) and the wild-type (normal) receptor PTK. Thus, in high-throughput screening and drug discovery compounds can be identified specifically targeting the mutant receptor without interfering with the normal receptor function. This allows the identification of highly specific compounds during the first primary screening step.

In yet another example this type of assay may be used to provide built-in controls for the compounds used in high-throughput screening. It is a common problem that compounds interfere with protein function in general rather than specifically with the function of the target protein resulting in a false positive signal. With the assay system provided by this invention the function of two different molecules with a common interacting partner may be monitored, for example the interaction of two different, independent GPCRs with their common downstream effector protein beta-arrestin. Therefore, the targeted interaction may be monitored by one RET pair (DT1-IG1:IG2-DT2). A second, related interaction may be monitored in parallel (DT1-IG1:IG3-DT3). Only compounds exhibiting an effect on the first pair but not on the second are target-specific. A compound with effects on both targets acts via an unspecific effect.

In yet another example this type of assay may be used to identify substances toxic for a particular organism but not another, i.e. a substance killing a parasite but not the host. A vital protein-protein interaction may be monitored with IGs derived from the parasite's proteins. In parallel the equivalent interaction with IGs derived from the host organism is monitored. The assay allows the identification of substances that are able to discriminate between the parasite's and the host's proteins.

Generally, for high-throughput screening, this type of assay can be used to find compounds specifically inhibiting or initiating one interaction (e.g. IG1:IG2) but not the other (IG1:IG3). This is important as the first interaction may cause a different cellular effect than the second, only one of which may be the desired effect of a drug. Therefore, this type of assay facilitates the development of drugs highly specific for a cellular effect. Also, this type of assay may be used to increase the throughput as two different interactions and functions are screened at the same time. This results in significant savings in reagents, cost and time.

It is clear to those skilled in the art that the aspects of molecular interaction as described above play an important role in numerous cellular functions and are not limited to those described in the examples.

(ii) Simple Detection System for Complex Molecular Associates—‘AND’ Assays.

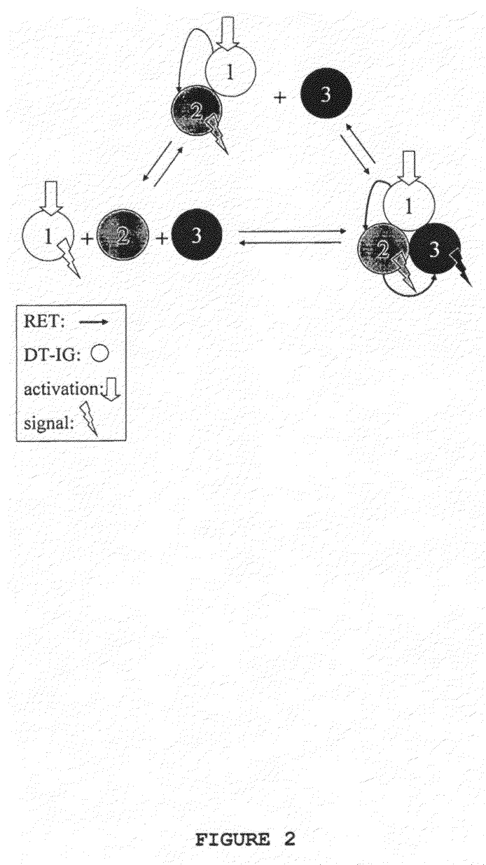

In one embodiment the emission spectrum of DT1 sufficiently overlaps with the excitation spectrum of DT2 but not DT3. The excitation spectrum of DT3 sufficiently overlaps with the emission spectrum of DT2 while the emission maxima of DT1, DT2 and DT3 are sufficiently distinct to allow their separate detection (FIG. 2 and Table 1). An example of a suitable combination of DTs is Renilla luciferase using a standard coelenterazine substrate (emitting at 460-480 nm) as DT1 in combination with EGFP or EYFP (DT2) and mRFP1 (DT3). Alternatively, EGFP or EYFP as DT2 may be substituted by the biarsenical dye FlAsH or a fluorescent moiety with similar spectral properties. Examples include Alexa Fluor 488 and Oregon green 514. The red fluorescent protein mRFP1 may be substituted by others fluorescent proteins or fluorescent moieties with similar spectral properties.

An example of an application of this embodiment is the monitoring of the activity of nuclear receptors which represent an important class of drug targets. In general, nuclear receptors dimerise upon binding of their ligand and then bind to DNA either activating or repressing transcription (Tsai, M. J. & O'Malley, B. W. (1994) Annu. Rev. Biochem. 63:451-486). According to this invention the subunits of the nuclear receptor dimer could be labelled with DTs as well as a double-stranded DNA fragment containing the binding site of the nuclear receptor complex. Upon activation of the receptor by a natural ligand or synthetic compound the trimeric protein-DNA complex is formed, the DTs are brought into proximity, and activating DT1 will result in a signal from DT3 indicating the formation of the associate and thus, activation of the receptor. Alternatively, the ligand or compound activating the receptor could be linked to a DT. Only if the compound induces dimerisation and thus, activates the receptor a signal from DT3 is obtained. Undesired compounds just binding to the receptor subunit without activating the receptor are therefore excluded. This distinction is not possible with current systems that detect molecular interactions.

In general this embodiment of the invention is useful when a simple answer on the formation on a complex molecular associate is desired and information about partially formed associates is not important. Many applications of this and the next embodiment overlap and which system is chosen depends on the required level of complexity of a particular application.

It is clear to those skilled in the art that the aspects of molecular interaction as described above play an important role in numerous cellular functions and are not limited to those described in the examples.

(iii) A Detection System for Complex Molecular Associates—‘Combinatorial’ Assays.

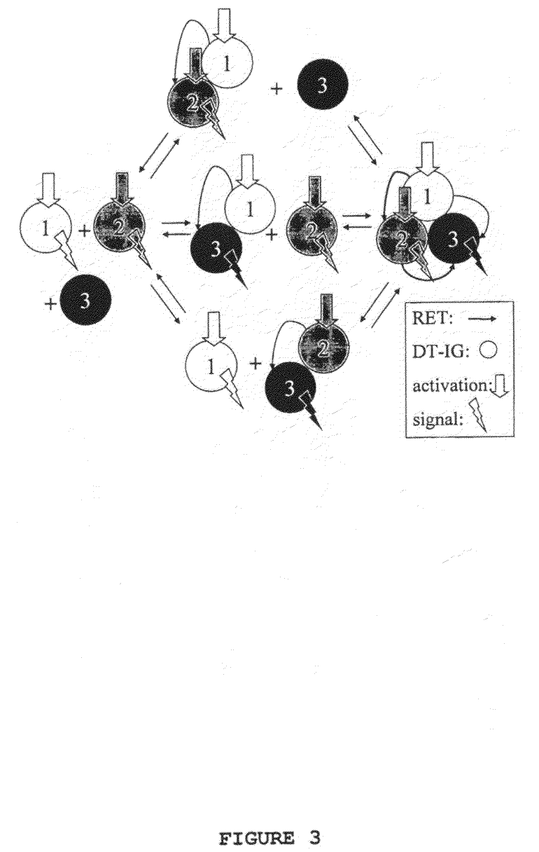

In another embodiment the emission spectrum of DT1 sufficiently overlaps with the excitation spectrum of DT2 and DT3, while the excitation spectrum of DT3 also sufficiently overlaps with the emission spectrum of DT2. Thus, DT1-DT2, DT1-DT3 and DT2-DT3 all form suitable RET pairs (FIG. 3 and Table 2). In contrast to the previous embodiments the detection in this embodiment occurs by sequentially activating DT1 by an appropriate substrate or energy source and detecting the emissions of DT1, DT2 and DT3 and then activating DT2 by an appropriate energy source and detecting the emissions of DT2 and DT3. Alternatively, DT3 may be a non-fluorescent quencher resulting in a decreased signal in DT1 and DT2 when RET occurs. The combined readout of the absence, presence or strength of the individual signals provides accurate information on the composition of the complex molecular associate (Table 2).

FRET systems involving three fluorescent moieties, all coupled to a short single-stranded oligonucleotide were reported recently (Tong et al. (2001) J. Am. Chem. Soc. 123, 12923-12924; U.S. Pat. No. 6,627,748; Haustein et al. (2003) Chemphyschem. 4, 745-748). Those DNA molecules act as probes with new fluorescent labels distinct from labels consisting only of single fluorescent moieties. FRET occurs from both the first and second fluorophore to the third fluorophore increasing the signal obtained from the third fluorophore. A similar system was applied to monitor conformational changes within a short double-stranded DNA molecule (Liu et al. (2002) J. Am. Chem. Soc. 124, 15208-15216). In the present invention however, three different labels combined with a series of activation and detection events are used to analyse a complex dynamic system of molecular interactions—which is not possible by these prior art systems.

Examples of suitable DTs include ECFP as DT1, EYFP as DT2 and mRFP1 as DT3. Alternatively, any of the DTs may be replaced by fluorescent moieties with similar spectral properties and which form suitable RET pairs with each other. In the above example mRFP1 could be replaced by Alexa Fluor 555 and could be used in conjunction with ECFP and EYFP.

One example for the application of this and the previous embodiment of the invention is the analysis of cytokine receptor signalling. Cytokine receptors form hetero-dimers of membrane-bound subunits when activated by binding of their ligand. One subunit is usually specific for the ligand whereas the other one is responsible for signal transduction and is shared by other ligand-specific subunits. The activated receptors interact with intracellular proteins like signal transducer and activator of transcription (STAT) proteins (Ishihara et al. (2002), Biochim. Biophys. Acta, 1592, 281-296). Thus cytokine receptor signalling involves a network of signal transducing molecules and receptor molecules with many overlapping and redundant functions. It is often difficult to attribute a particular effect to the actions of specific molecules or receptors. IGs can be derived from receptor subunits forming a suitable RET pair (DT1-IG1:IG2-DT2) when the receptor is activated and dimerises.

TABLE 2
Assays involving non-fluorescent quenchers as DTs and
expected signals upon activation of DTs by an appropriate
substrate or excitation light. Numbers indicate an
increased signal of this DT, dashes indicate a decreased
signal/no activation of other DTs.

Activation of

| | | DT1 | DT2 |
|---|---|---|---|
| Assay type | IG association | activates | activates |
| detection system for | none | 1 | 2 |
| complex molecular | 1:2 | 1 + 2 | 2 |
| associates | 1:3 | 1 + 3 | 2 |
| | 2:3 | 1 | 2 + 3 |
| | 1:2:3 | 1 + 2 + 3 | 2 + 3 |
| detection system for | none | 1 | 2 |
| complex molecular | 1:2 | 2 | 2 |
| associates using a | 1:3 | — | 2 |
| non-fluorescent | 2:3 | 1 | — |
| quencher as DT3 | 1:2:3 | — | — |

To further monitor the activation of a particular signal transducer by the receptor, a third IG is derived from the signal transducing protein (IG3-DT3). When this molecule interacts with the activated receptor complex energy is transferred from DT2 to DT3 and light of a specific wavelength is emitted from DT3 or the signal from DT2 and/or DT1 is quenched. This signal is specific for the activation of this particular pathway by this particular cytokine receptor. Activation of another pathway by the same cytokine receptor yields a signal for the receptor activation but not the particular signalling pathway. Activation of the same pathway via another cytokine receptor does not give a signal.

Another example is the analysis of G-protein coupled receptors (GPCRs) that form homo or hetero-dimers. Recent studies have shown that GPCRs may not only act as monomers but also as homo- and hetero-dimers which causes altered ligand binding, signalling and endocytosis (Rios et al. (2000) Pharmacol. Ther. 92, 71-87). The effect of drugs acting as agonists or antagonists of a specific receptor may therefore depend on the binding partners of this receptor. It may be desirable to limit the effect of a drug to a cellular response mediated by a specific receptor dimer. The system provided by this invention monitors the activity of a specific GPCR dimer. The GPCRs themselves act as IGs and are attached to DTs (IG2-DT2, IG3-DT3). A third IG (IG1-DT1) is derived from a molecule that interacts with GPCRs upon ligand binding (e.g. β-arrestin). The detection system not only detects the formation of the receptor heterodimer but can distinguish whether a ligand or drug activates (or blocks) the receptor heterodimer, the respective homodimers or a combination thereof.

Another example is the transcriptional regulation of gene expression. Transcription factors act in multiprotein-DNA complexes and the composition of these complexes determines their specificity and activity (Wolberger et al. (1999) Ann. Rev. Biophys. Biomol. Struct. 28, 29-56). For example the transcription factor Fos is only active as a hetero-dimer with a member of the Jun transcription factor family (Chinenov et al. (2001) Oncogene 20, 2438-2452). The Fos/Jun dimer can activate or repress the transcription of numerous genes. The specificity and activity of the complex is regulated by additional proteins interacting with the dimer, like ETS transcription factors, NF-AT or Smad proteins (Wang et al. (1994) Mol. Cell Biol. 14, 1153-1159; Stranick et al. (1994) J. Biol. Chem. 272, 16453-16465; Zhang et al. (1998) Nature 394, 909-913). IGs can be derived from Fos and Jun proteins attached to DTs forming a suitable RET pair (DT1-IG1:IG2-DT2). This RET signal indicates a functional dimer of a particular Fos/Jun combination. The third IG is derived from a transcriptional regulator interacting with the Fos/Jun complex. This IG is attached to a third DT (IG3-DT3) that emits or quenches light transferred from DT2 when IG3 interacts with the IG1:IG2 complex. This signal is specific for the activity of the trimeric complex involving a particular combination of Fos/Jun proteins. Activation of Fos/Jun by interaction with other regulators or activation of different Fos/Jun complexes with the same regulator will result in different signals.

Another example is the development of novel antiviral drugs. A major problem of therapies for HIV and other viruses is the adaptability of the virus by point mutations of viral proteins to gradually become resistant to all drugs being developed so far. Therapies that target multiple events in the viral life cycle are therefore more successful, and mixtures of different drugs, so-called combination therapies have found wide clinical use. Promising, novel anti-retroviral drugs are virus entry inhibitors (Starr-Spires et al. (2002), Clin. Lab. Med. 22, 681-701). The entry of HIV virions is mediated via two cellular receptors: CD4 and CXCR4 or CCR5, depending on the virus strain. Antibodies or drugs only blocking the virus-CD4 interaction rapidly loose their efficiency as the viral surface changes. The system provided by this invention allows the simultaneous detection of the viral binding to both receptors. The two receptors plus the viral surface protein can be labelled with DTs yielding a specific signal when the trimeric complex is formed. Thus, compounds can be identified that efficiently block both interactions or inhibit required conformational changes of the viral protein to bind to both receptors. As two vital interactions are targeted simultaneously the emergence of resistant viruses is less likely.

In another example, the invention is used to analyse the composition, conformation, assembly or dissociation of a large, stable molecular complex. The presence or absence of the different RET signals indicates the assembly and functionality of the complex or conformational changes/movements of within the complex or components of the complex. Examples of complexes include transcription factor complexes, ribosomes, proteasomes, chaperones, oligomeric receptors, ion channels etc.

Generally, for high-throughput screening and drug discovery, this type of assay can be used to find compounds inhibiting or activating the function of a molecule in its environment within a specific multi-component molecular associate. The function of the same molecule within another associate may not be affected.

It is clear to those skilled in the art that the aspects of molecular interaction as described above play an important role in numerous cellular functions and are not limited to those described in the examples.

The term “detecting emitted light” as used herein refers to any detection device capable of detecting photons of a specific wavelength in a quantitative manner. Examples include photomultiplier tubes or CCD cameras. The detector further comprises a means of restricting the detected light to a specific wavelength or a specific range of wavelengths. This can be for example suitable filters mounted to a filter wheel or a filter slide or a monochromator.

In one embodiment the first DT is activated by excitation light specific for this DT and the light emitted by this DT and the other DTs is detected. Then the second DT is activated and the emitted light of this and other DTs is detected and so forth. The combined information provided by these sequential readings provides information on the associations between the IGs as summarised in Table 2. This sequence of activation and detection may be repeated in time intervals to obtain kinetic data. At any time of this detection sequence substances can be added or conditions may be changed that may influence the associations of the IGs.

In another embodiment a suitable substrate is added for the activation of a first DT. The emitted light of this DT and the other DTs is detected while the excitation light is turned off or blocked. Then, excitation light specific for the activation of a second DT may be turned on and the emitted light of this DT and the other DTs is detected. To obtain kinetic data the detection mode can be switched continually between luminescence and fluorescence detection with the light source turned off and on, respectively. At any time of this detection sequence substances can be added or conditions may be changed that may influence the associations of the IGs.

In yet another embodiment a substrate suitable for a first DT is added. The emitted light of this DT and the other DTs is detected while the excitation light is turned off or blocked. When this first substrate is used up and the light emission from the first DT ceased, a second substrate suitable for a second DT is added. The emitted light of this DT and the other DTs is detected while the excitation light is turned off or blocked. At any time of this detection sequence substances can be added or conditions may be changed that may influence the associations of the IGs.

The invention will now be further described by way of reference only to the following non-limiting examples. It should be understood, however, that the examples following are illustrative only, and should not be taken in any way as a restriction on the generality of the invention described above. In particular, while the invention is described in detail in relation to the use of specific tags and interacting groups, it will be clearly understood that the findings herein are not limited to these tags or interacting groups.

### EXAMPLE 1 ANALYSIS OF PROTEINACEOUS FRET PAIRS

For assays according to this invention it is important to have tags with sufficient spectral overlap to from a RET pair and others that are separated enough that no RET occurs. The efficiency of RET can be described by the Foerster radius R₀. R₀ is the distance at which energy transfer is 50% efficient, ie. 50% of excited donors are deactivated by FRET. The magnitude of R₀ is mostly dependent on the spectral properties of the donor and acceptor dyes:

R₀=└8.8×10²³*κ²*n⁻⁴*QY_{D}*J(λ)┘^{1/6}\dot{A}

where

κ²=dipole orientation factor (range 0 to 4; ⅔ for random orientation)

QY_{D}=fluorescence quantum yield of donor in the absence of acceptor or luminescence capacity of a bioluminescent protein

n=refractive index (1.33 for water, depends on temperature, ionic strength)

J(λ)=spectral overlap integral

To adjust the efficiency of RET the selection of dyes with a high quantum yield and sufficient spectral overlap (i.e. J(λ) is large) is the most important variable. Assays according to this invention occur in an aqueous medium suitable for biological molecules, therefore there is little variation in the refractive index n. The geometric orientation of the dyes, i.e. the dipole orientation factor κ², will be near ⅔ in most situations, the value for randomly orientated molecules. This is because the tagging occurs with flexible linkers and spacing groups that allow the dyes more or less free rotation relative to the attached interacting group, although the use of bulky fluorescent proteins as DTs may limit the rotational freedom.

Consequently, the spectral properties of existing fluorescent proteins and their use as DTs according to this invention were examined. As a simple model system for interactions to investigate potential RET, fusion proteins of proteinaceous DTs were generated. This is the most ideal, permanent interaction and therefore well suited to define the magnitude of which RET can occur. The DT subunits were separated by linkers which had lengths of 7-18 amino acids. The linker sequences contained mostly serine and glycine residues for maximum flexibility. This allowed free rotation of the subunits against each other and prevented a loss of the RET signal due to an unfavourable geometric orientation.

The coding sequences of mRFP1, t-dimer2(12), ECFP, EGFP and EYFP were amplified via PCR with the following oligonucleotides (Table 3): mRFP1-fw/re, template: pRSETB-mRFP1 (Campbell et al., (2002), PNAS. USA, 99, 7877-7882); t-dimer2(12): mRFP1-fw/t-dimer2(12)-re, template: pRSETB-t-dimer2(12) (Campbell et al. (2002), PNAS. USA, 99, 7877-7882; EGFP-P3-fw/re, template: pECFP-N1 (Clontech); EGFP-P2-fw/re, template:

TABLE 3
OLIGONUCLEOTIDE SEQUENCES

| Oligo | Sequence |
|---|---|
| mRFP1-fw | GACGATGACGATAAGGATCCGATG |
| mRFP1-re | CTTCGAATTCGAGGCGCCGGT |
| t-dimer2(12)-re | TCAAGCTTCGAATTCGACAGGAAC |
| EGFP-P2-fw | TATAGAGCTCGGTGAGCAAGGGCGAGGAGCTG |
| EGFP-P2-re | ATATAGTCGACCTTGTACAGCTCGTCCATGCCG |
| linker-12-fw | AATTCTGGCAGCGGTTCCGGCTCTGGTAGCT |
| linker-12-re | ACCAGAGCCGGAACCGCTGCCAG |
| Rluc-P3-fw | TAAAATTGCGGCCGCTTCCAAGGTGTACGACCCCGA |
| Rluc-P3-re | TATACTTAAGTTACTGTTCGTTCTTCAGCACGC |
| EGFP-P1-fw | TAGGATCCGGTGAGCAAGGGCGAGGAGCTG |
| EGFP-P1-re | TAGAATTCCCCTTGTACAGCTCGTCCATGCCG |
| linker-18-fw | AATTCTGGCAGCGGTTCCGGCTCTGGTTCTGGCAGCGGTAGCGGTAGCT |
| linker-18-re | ACCGCTACCGCTGCCAGAACCAGAGCCGGAACCGCTGCCAG |
| Rluc-P1-fw | TAGGATCCGGCTTCCAAGGTGTACGACCCCGA |
| mRFP1-P3-re | TATACTTAAGTTAGGCGCCGGTGGAGTGGC |
| t-dimer2(12)-P3-re | TATACTTAAGTCACAGGAACAGGTGGTGGCGGC |
| MCS-linker-fw | CTAGCCGCCACCATGGTAAGCTTCTGCC |
| MCS-linker-re | TCGAGGCAGAAGCTTACCATGGTGGCGG |
| CCR2-fw | TAATAAAGCTTCCTGTCCACATCTCGTTCTCGG |
| CCR2-re | ATTGGATCCCCTAAACCAGCCGAGACTTCCTGC |
| EGFP-P3-re | TATACTTAAGTTACTTGTACAGCTCGTCCATGCC |
| Rluc-fw | CGACTCACTATAAGCTTGCCACCATGAC |
| Rluc-re | GCCGCTCTAGATATCTTGTTCATTTTTG |
| Barr2-fw | AAAGATATCATGGGGGAGAAACCCGG |
| Barr2-re | TATATGCGGCCGCCACTAGCAGAACTGGTC |

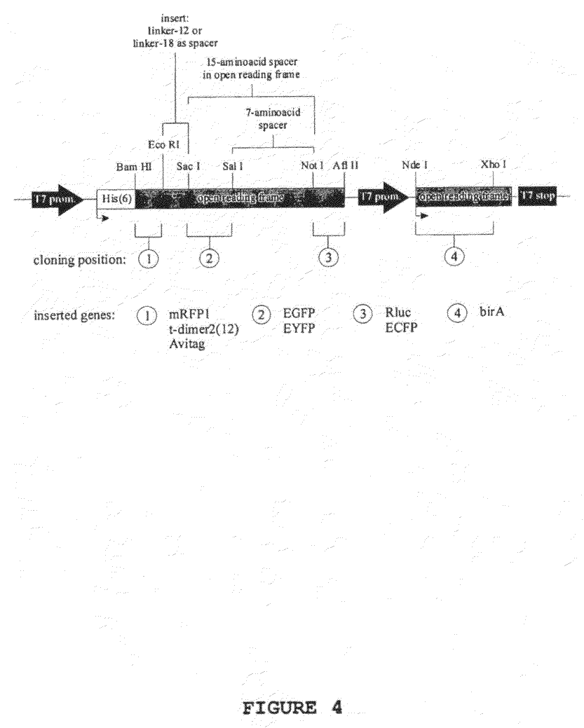

pEGFP-N1 (Clontech); EGFP-P2-fw/re, template pEYFP-N1 (Clontech). The ends of the products were cut with the appropriate restriction enzymes and cloned into the vector pETDuet-1 as outlined in FIG. 4. If required, oligonucleotide linkers, encoding the peptide spacers, were inserted between the subunits. This resulted in the following constructs: pET-mRFP1, pET-t-dimer2(12), pET-ECFP, pET-EGFP, pET-EYFP, PET-EYFP-ECFP and pET-t-dimer2(12)-12-ECFP where the number between the subunits describes the length and position of the linker (FIG. 4).

E. coli Rosetta cells (Novagen) were transformed by these plasmids and grown in 100 ml cultures at 37° C. until an OD₆₀₀=0.7 was reached. A total of 0.5 mM IPTG was added, and the cultures were incubated in a shaker at 20° C. over night. The cells were harvested by centrifugation for 30 min at 3500×g. One half of the cell pellet was frozen at −80° C. for later use. The other half was lysed with 800 μl BugBuster reagent (Novagen) following the manufacturer's instructions. The proteins were purified from the clear lysate via their N-terminal His-tags using 300 μl HisMag magnetic beads (Novagen) according to the manufacturer's instruction. The spectral properties of the proteins were determined with a Cary Eclipse fluorescence spectrometer (Varian).

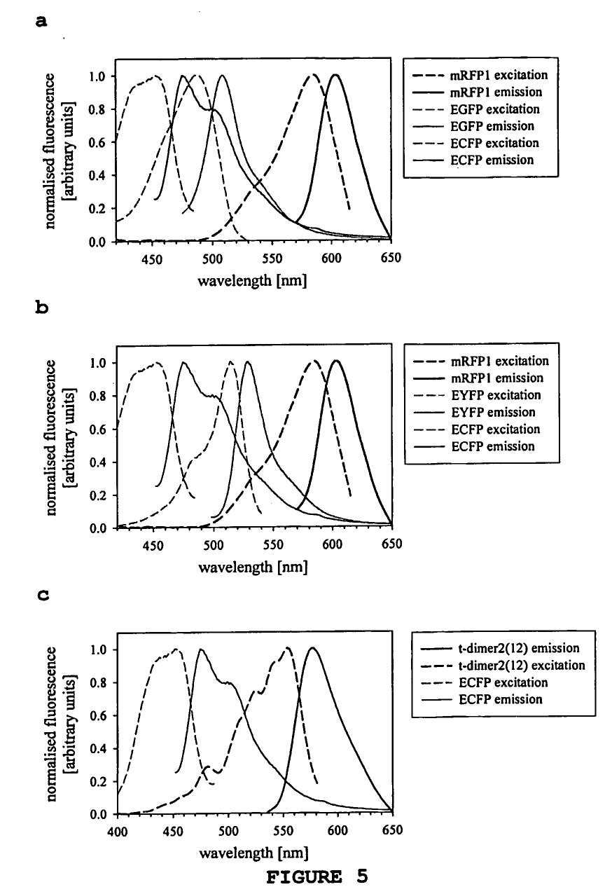

The spectral properties and spectral overlap of mRFP1, t-dimer2(12), ECFP, EGFP and EYFP are shown in FIG. 5. All spectra were normalised to their maximum excitation and emission (arbitrary value “1”). ECFP and EGFP show large spectral overlap but poor distinction between their excitation spectra whereas the mRFP1 excitation has only little overlap with either fluorescence emission (FIG. 5a). The well-characterised FRET pair ECFP-EYFP shows significant overlap between donor emission and acceptor excitation, while the donor and acceptor excitations are sufficiently separated. EYFP also overlaps well with the mRFP1 excitation suggesting that EYFP and mRFP1 are able to form a suitable RET pair (FIG. 5b). ECFP and t-dimer2(12) show a surprisingly large spectral overlap despite the large separation of their emission maxima, indicating the potential formation of a suitable RET pair with an emission that is spectrally distinct from the ECFP-EYFP pair (FIG. 5c).

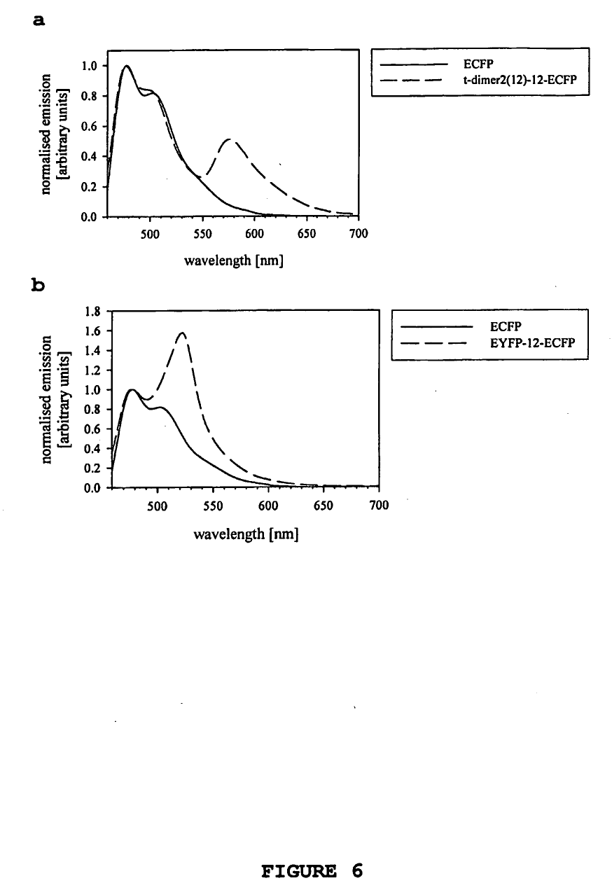

Next, RET between the subunits of the fusion proteins was analysed (FIG. 6). The EYFP-12-ECFP and t-dimer2(12)-12-ECFP fusion proteins were excited at 440 nm and the emission was scanned between 460 and 700 nm. The same scan was performed with EYFP and t-dimer2(12) proteins. A further scan was performed on an empty well which was subtracted as background from the fusion protein and fluorescent protein spectra. The spectra were further corrected for light emission due to direct excitation of the acceptor fluorophores by the light source by subtracting the EYFP spectrum from the EYFP-12-ECFP spectrum and the t-dimer2(12) spectrum from the t-dimer2(12)-12-ECFP spectrum after all spectra were normalised to their acceptor fluorophore emission using excitation light of a longer wavelength that does not excite the donor fluorphores, i.e. 490 nm for EYFP and 540 nm for t-dimer2(12). Finally the corrected FRET spectra were normalised to the arbitrary value “1” at the ECFP emission maximum at 480 nm to allow for a comparison of the data (FIG. 6). A significant FRET signal was observed for both EYFP and t-dimer2(12) as acceptor fluorophores. This is remarkable as the t-dimer2(12) fluorescent protein contains effectively two fluorescent groups doubling any direct excitation background signal while only one fluorescent group will contribute significantly to a RET signal, because of being physically closer to the donor DT. Therefore, the detection system could be further improved by using a true monomeric fluorescent protein or a non-proteinacious fluorophore with similar spectral properties as t-dimer2(12).

### EXAMPLE 2 ANALYSIS OF PROTEINACEOUS BRET PAIRS

The use of bioluminescent proteins as DTs requires fluorescent DTs with sufficient spectral overlap to the luminescence emission. The potential for RET between Renilla luciferase (Rluc) as a bioluminescent protein DT and the proteinaceous DTs EGFP, EYFP, t-dimer2(12) and mRFP1 was investigated. Again, as a simple model system for interactions fusion proteins between Rluc and proteinaceous DTs were generated.

The gene for Rluc was amplified via PCR with the following oligos: Rluc-P3-fw/re, template phRL-CMV (Promega). Using appropriate restriction enzymes the PCR product was cloned into the vectors from Example 1 resulting in the constructs pET-Rluc, pET-EGFP-15-Rluc, pET-EYFP-7-Rluc, pET-t-dimer2(12)-15-Rluc and pET-mRFP1-15-Rluc, where the number between the subunits describes the length and position of the linker (FIG. 4).

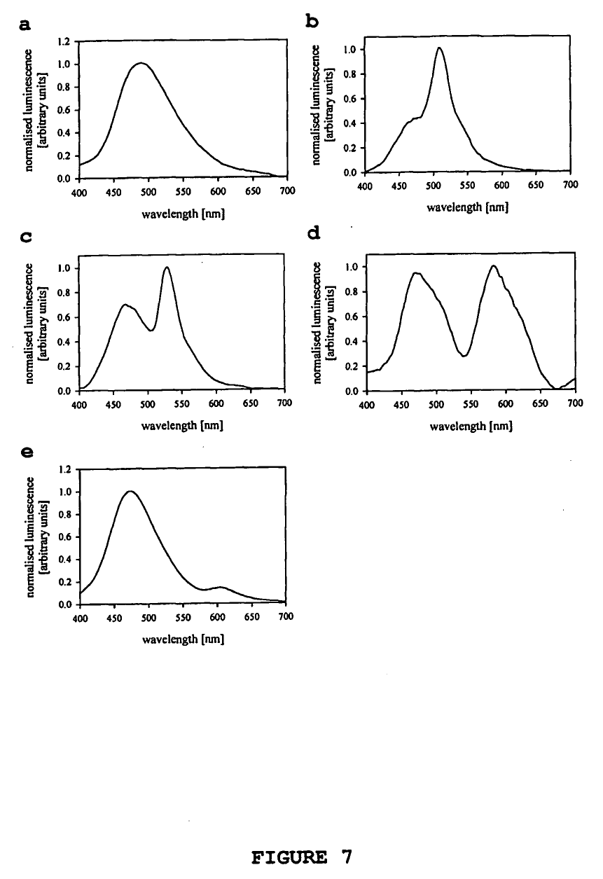

The proteins were expressed in E. coli Rosetta cells and purified as described in Example 1. Luminescence spectra were recorded with a Cary Eclipse (Varian) luminescence spectrometer after the addition of 5 μM coelenterazine h as a substrate for Rluc (FIG. 7). The spectra were normalised to their emission maxima (arbitrary value “1”). Additional peaks occurring compared to Rluc are due to RET and were observed with all acceptor DTs.

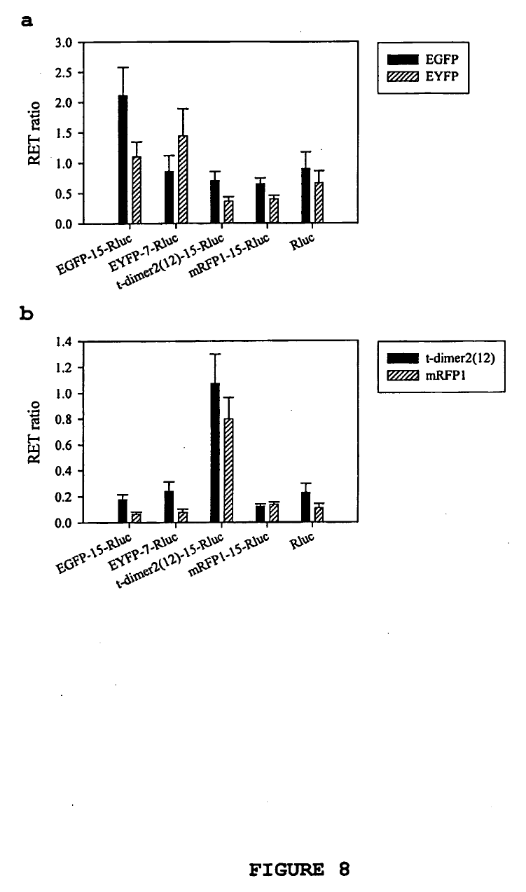

From the spectra RET ratios (RR) were calculated as a measure for the RET (FIG. 8). RET ratios are also an indicator for the distance and the orientation between the DTs. The ratios were calculated as follows:

The emission peaks were integrated in 10-20 nm windows covering the emission maxima of the DTs by adding up the emission values within this range.

Simple RET ratios (RR) for the yellow and red channels were calculated:

RR^{Y}=yellow/blue

RR^{R}=red/blue

with blue being the peak area under the Rluc emission peak, yellow being the peak area under EGFP or EYFP emission peaks and red being the peak area under the t-dimer2(12) or mRFP1 emission peaks. If the spectra are normalised to the first (donor) DT emission maximum prior to the integration then the values of the peak areas under the acceptor DTs and the RET ratios provide identical results in relative terms.

Normalised RET ratios (RRₙₒᵣₘ) are ratios corrected for the signal obtained by the energy donor alone.

RR^{Y}ₙₒᵣₘ=RR^{Y}−RR^{Y}₀; RR^{Y}₀=yellow₀/blue₀

RR^{R}ₙₒᵣₘ=RR^{R}−RR^{R}₀; RR^{R}₀=red₀/blue₀

with blue₀, yellow₀ and red₀ being the areas under the equivalent peaks of the first (donor) DT in the absence of an interaction and RET with another DT.

The results (FIG. 8) showed that the t-dimer2(12) emission could be surprisingly well distinguished from either EGFP or EYFP emissions while achieving a better signal-to-noise ratio than the standard EYFP acceptor DT. EGFP and EYFP as well as t-dimer2(12) and mRFP1 could not be separated clearly and showed significant ‘leakage’ between the respective channels. Although the mRFP1 emission was separated by 100 nm from EGFP the signal separation was still poor because Rluc cannot substantially activate mRFP1 and as a consequence only a weak red fluorescence signal is detected.

### EXAMPLE 3 ANALYSIS OF NON-PROTEINACEOUS BRET PAIRS

The choice of DTs is not restricted to proteinaceous molecules. Small fluorescent molecules as DTs may for many applications offer advantages as they are available with a wider range of spectral properties, and their smaller size makes them less likely to interfere with the function of the attached interacting group. As a model system for biological interactions the strong affinity of the protein streptavidin to the biotin-group was used. Streptavidins are available as conjugates with many different small molecule fluorescence dyes.

The E. coli enzyme biotin ligase (birA) mediates in the presence of ATP the attachment of a biotin-group to a lysine-residue of a specific 13-aminoacid peptide sequence called ‘avitag’ (Schatz, (1993), Bio/Technology, 11, 1138-1143). The coding sequence of the avitag was inserted into the construct pET-Rluc as a linker consisting of the hybridised oligonucleotides AvitagN-fw/re (Table 3). The linker was cloned into position 1 (FIG. 4) of this construct. The coding sequence for biotin ligase was amplified with the oligonucleotides birA-fw/re using E. coli Top10 as a template. The birA gene was cloned into position 4 of the construct (FIG. 4) to co-express biotin ligase for a quantitative biotinylation of the target sequence. The resulting construct for the expression of a biotinylated Rluc protein was called pET-Avi-15-Rluc/birA.

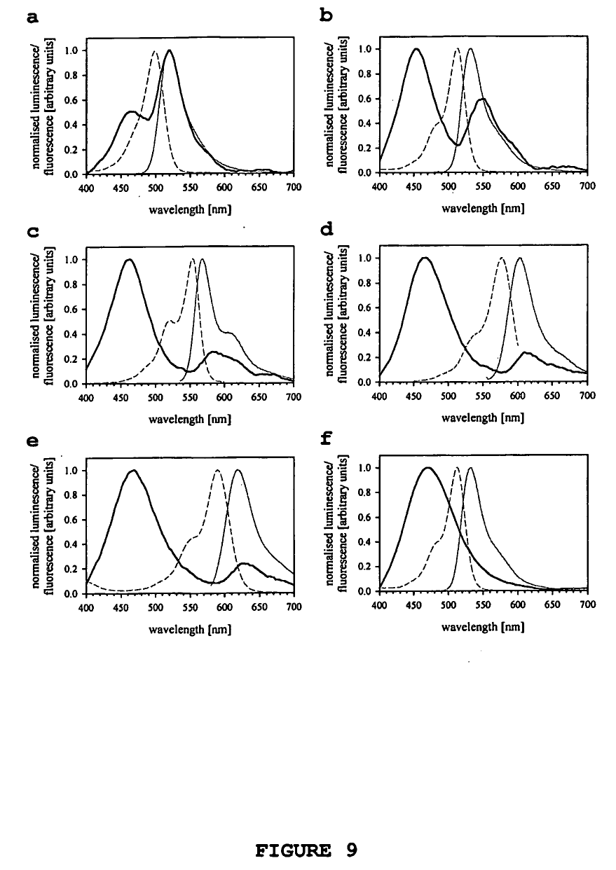

The protein was expressed in E. coli Rosetta cells and purified as described in Example 1. To the biotin-Rluc protein solution BSA was added to a final concentration of 2 mg/ml to prevent an unspecific interaction of the proteins. An approximately equimolar amount of streptavidin conjugates was added to the solution and luminescence spectra were recorded with a Cary Eclipse (Varian) luminescence spectrometer after the addition of 5 μM coelenterazine h as a substrate for Rluc (FIG. 9). The spectra were normalised to their emission maxima (arbitrary value “1”).

All small fluorescence dyes were suitable as DTs and resulted in RET signals in the presence of the substrate coelenterazine h (FIG. 9). Alexa Fluor 488 had the biggest spectral overlap with the Rluc emission and thus showed the highest RET signal. Surprisingly however, Alexa Fluor 594, the spectrally most distant dye used, resulted in a RET signal of similar or greater magnitude than the spectrally more overlapping dyes Alexa Fluor 555 and Alexa Fluor 568. Non-biotinylated Rluc was used as a negative control and did not yield a RET signal in the presence of a streptavidin conjugate at a similar concentration, verifying the specificity of the biotin-streptavidin model interaction.

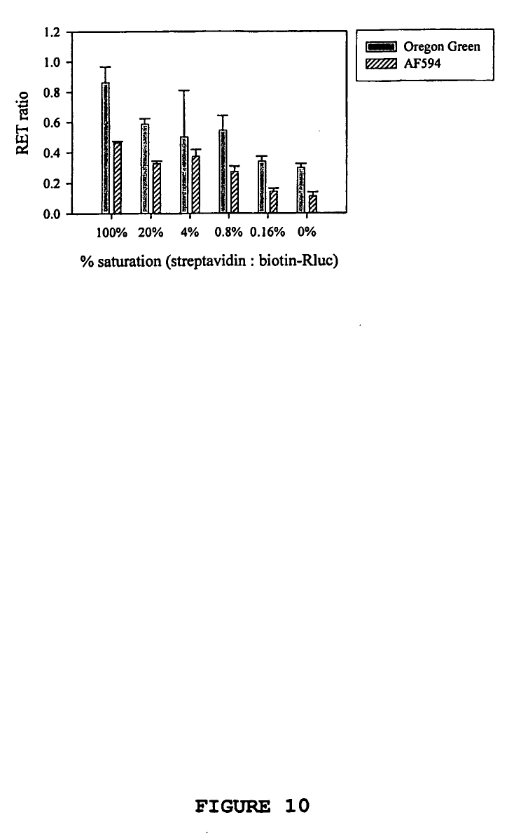

An important aspect of assays according to this invention is that they provide a quantitative and sensitive measure for biological interactions. Serial dilution of streptavidin conjugates were incubated with biotinylated Rluc plus 2 mg/ml BSA. After the addition of 5 μM coelenterazine h, luminescence spectra were recorded, and the RET ratios were calculated using the equations from Example 2 with adjustment of the integration range to the emission maxima of the respective dyes. FIG. 10 shows that for both conjugates, Oregon green and Alexa Fluor 594, the RET ratio correlated with the streptavidin conjugate concentration, demonstrating that the detection system provided a quantitative measure for interactions. It was also remarkable that Alexa Fluor 594, despite its little spectral overlap with the Rluc emission was as sensitive as the much more overlapping Oregon green in detecting the biotin-streptavidin interaction.

### EXAMPLE 4 SIMULTANEOUS, MULTIPLEX RET DETECTION

According to this invention multiple biological interactions can be detected simultaneously and in a quantitative manner (‘multiplexing’). To explore this possibility the model systems used in Examples 2 and 3 were tested for their multiplexing capabilities.

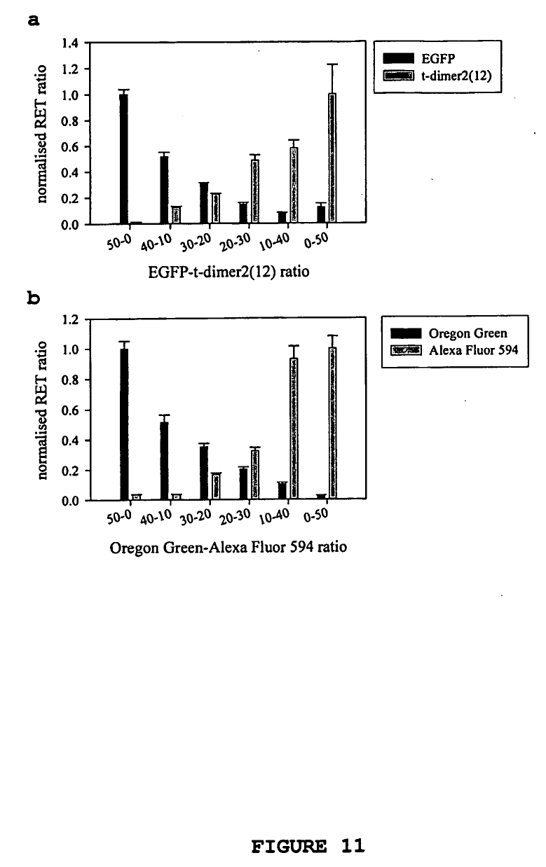

In Example 2, the proteinaceous DTs, EGFP and t-dimer2(12) were identified as potential DTs in multiplex combinations due to the sufficient spectral resolution between their emission maxima. The concentrations of the EGFP-15-Rluc and t-dimer2(12)-15-Rluc fusion proteins were adjusted to similar concentrations. The protein solutions were mixed stepwise with ratios of 5:0, 4:1 . . . 1:4, 0:5. The Rluc substrate coelenterazine h was added to a final concentration of 5 μM and luminescence spectra were recorded. RET ratios were calculated and normalised to the highest value (arbitrary value “1”) for an easier comparison of the two channels (FIG. 11a).

Multiplex detection was further tested using the biotin-streptavidin interaction model. Biotinylated Rluc plus 2 mg/ml BSA was mixed with an equimolar amount of streptavidin-Oregon green in one tube and with streptavidin-Alexa Fluor 594 in a separate tube. The solutions were mixed stepwise with ratios of 5:0, 4:1 . . . 1:4, 0:5. The Rluc substrate coelenterazine h was added to a final concentration of 5 μM, and luminescence spectra were recorded. RET ratios were calculated and again normalised to the highest value (arbitrary value “1”) for an easier comparison of the two channels (FIG. 11b).

In both experiments true multiplexing was achieved: the signal of both channels was detected independently, providing accurate information on the interactions within the different complexes and mixtures.

In summary, this example demonstrates that this invention enables multiplex detection of biological interactions in a variety of applications and embodiments. This has been shown to be independent of the model used and independent of the nature of the DT.

### EXAMPLE 5 MULTIPLEX DETECTION IN MAMMALIAN CELLS (1)

An important aspect of the function of G-protein coupled receptors (GPCRs) is their ability to form homodimeric complexes. In this example the assay system of the present invention was tested to detect multiple receptor-receptor interactions. As receptors are membrane proteins their functional extraction and purification is often difficult or impossible. Therefore, an assay system according to this invention was also used in live, mammalian cells.

The oligonucleotides MCS-linker-fw and MCS-linker-re (Table 3) were hybridised and cloned into the Nhe I and Xho I restriction sites of the plasmid pcDNA3.1(−) (Invitrogen), resulting in the vector pcDNA-MCS. The linker provided an optimal Kozak sequence for the high level expression of proteins as well as a translational start site and the beginning of an open reading frame.

The cDNAs encoding the fluorescent proteins ECFP, EYFP and t-dimer2(12) were amplified by PCR using the oligos EGFP-P1-fw/EGFP-P3-re (EGFP or EYFP) and mRFP-fw/t-dimer2(12)-P3-re (t-dimer2(12) as described in Example 1. The PCR products were cloned into the plasmid pcDNA-MCS resulting in the vectors pcDNA-MCS-ECFP, pcDNA-MCS-EYFP, pcDNA-t-dimer2(12), each being capable to express the fluorescent protein in mammalian cells. The cDNA encoding CCR2 receptor was amplified by PCR using the oligos CCR2-fw/CCR2-re. The cDNA template was obtained from the Guthrie Research Institute (USA). The PCR product were cloned 5′ of the fluorescent proteins into the plasmids pcDNA-MCS-ECFP, pcDNA-MCS-EYFP and pcDNA-MCS-t-dimer2(12) to assemble expression constructs of C-terminal fusions of the receptor with a fluorescent protein. The TRHR GPCR cDNA was excised from the pcDNA3-TRHR/Rluc vector (Kroeger et al., (2001), J. Biol. Chem., 276: 12736-12741) using the restriction enzymes Hind III and Not I. This fragment was cloned into the pcDNA-MCS-ECFP, pcDNA-MCE-EYFP and pcDNA-MCS-t-dimer2(12) vectors. The resulting final plasmids were named pcDNA-CCR2-ECFP, pcDNA-CCR2-EYFP, pcDNA-CCR2-t-dimer2(12), pcDNA-TRHR-ECFP, pcDNA-TRHR-EYFP, pcDNA-TRHR-t-dimer2(12).

Cos-7 cells, an adherent mammalian cell line, were used to express the receptor fusion constructs. The cells were grown to a density of about 60% confluence under standard culture conditions. The cells were transfected by one or a combination of several expression vectors using Genejuice (Novagen) transfection reagent according to the manufacturer's protocol. After 2 days incubation under standard culture conditions the cells were trypsinised, and the concentration of the cell suspension was adjusted to about 50000 cells in 50 μl PBS.

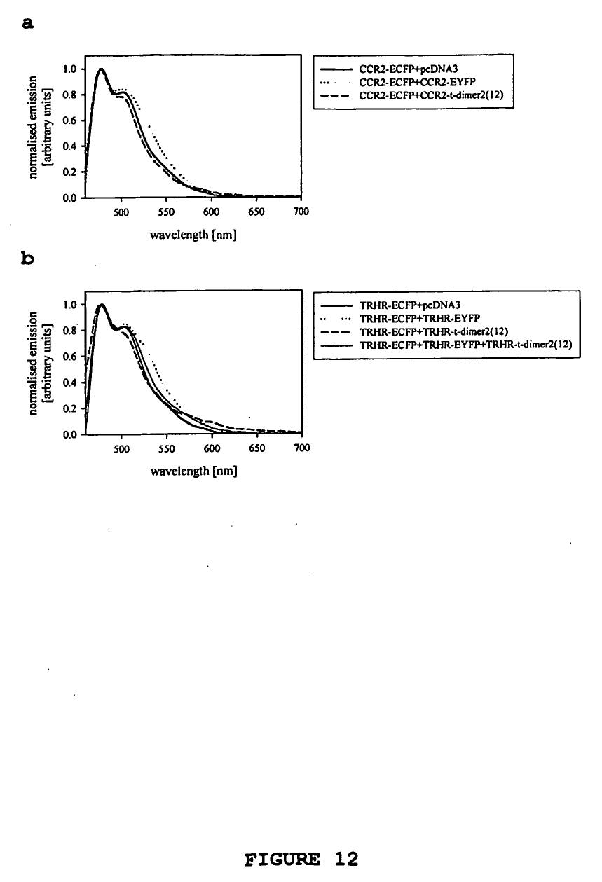

These cell suspensions were analysed by fluorescence spectroscopy using a Varian Eclipse fluorescence spectrometer (Varian). The samples were excited at 440 nm, the excitation maximum of ECFP, and the fluorescence emission was recorded between 460 and 700 nm. Emission due to direct excitation of the acceptor fluorophores (EYFP or t-dimer2(12)) was subtracted from the spectra as described in Example 1. The corrected spectra were further normalised to the emission maximum of ECFP (arbitrary value “1”) and compared to a sample expressing only a receptor-ECFP fusion protein (FIG. 12). The homodimer formation was detected for both receptors by both RET pairs. The homodimer formation could be detected individually or simultaneously (FIG. 12b).

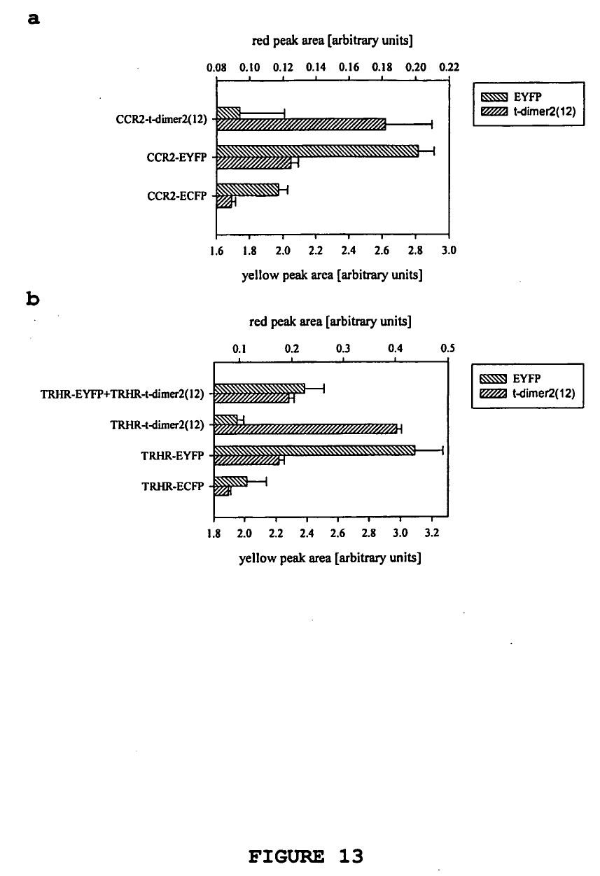

To quantitate the results, the spectra were integrated and the peak areas of the acceptor peaks were calculated as described in Example 2. FIG. 13 shows that the yellow and red channels can be detected independently as required for a multiplex system. It further shows that within the TRHR homodimer complex the interactions were stronger or the DTs were arranged in a more favourable orientation compared to the CCR2 complex. Despite the weaker interaction, the CCR2 homodimer interaction was readily detected by using either EYFP or t-dimer2(12) as a RET acceptor (FIG. 13a). When two homodimer interactions were detected simultaneously the absolute signals were lower than each of the individual signals but still above the background signals (FIG. 13b). This was due to the transient transfection system used here resulting in a lower number of triple versus double co-transfected cells.

In summary this example demonstrates that the detection system described in this invention provides a useful assay for a quantitative, multiplex detection of interactions among membrane proteins in live mammalian cells. It is obvious that instead of live cells membrane preparations or membrane fractions could be used for the analysis of membrane proteins.

### EXAMPLE 6 MULTIPLEX DETECTION IN MAMMALIAN CELLS (2)

Cell signalling involves the interaction of cytoplasmic proteins with membrane bound cell surface receptors. For GPCRs, the interaction with the cytoplasmic protein beta-arrestin-2 after activation by a ligand plays an important role for the receptor desensitisation and internalisation. In this example, the activation of different receptors was tested in a multiplex assay system to detect the receptor beta-arrestin-2 interactions. This was done in live mammalian cells as the receptors after ligand binding have to be modified by cellular kinases in order to interact with beta-arrestin-2. Also, an isolation of functional, membrane bound receptors is difficult to achieve.

A construct for the expression of bovine beta-arrestin-2, N-terminally fused to Rluc (Rluc-Barr2) was constructed by amplification of the bovine beta-arrestin-2 coding sequence by PCR using the Barr2-fw and Barr2-re primers (template: construct containing bovine beta-arrestin2 in pcDNA3 (Invitrogen)). The 5′ and 3′ primers used contained EcoRV and NotI restriction enzyme sites, respectively. A second PCR product, containing the coding sequence for Rluc, was generated by amplifying the Rluc cDNA sequence with the primers Rluc-fw and Rluc-re using the plasmid pRL-CMV (Promega) as a template. This second PCR product contained HindIII and EcoRV restriction site at the 5′ and 3′ ends, respectively. Both PCR products were then cloned together into the HindIII/NotI sites of pcDNA3 (Invitrogen). The resulting plasmid, for the mammalian expression of an Rluc-beta-arrestin-2 fusion protein was named pcDNA-Rluc-Barr2.

The mammalian cell line Cos-7 was simultaneously transfected by three plasmids, either pcDNA-Rluc-Barr2, pcDNA-TRHR-EYFP and pcDNA-CCR2-t-dimer2(12) or pcDNA-Rluc-Barr2, pcDNA-TRHR-t-dimer2(12) and pcDNA-CCR2-EYFP. Transfections were performed using Genejuice (Novagen) according to the manufacturer's instructions. After transfection, cells were cultured for two days under standard conditions. The cell were then trypsinised, and a cell suspension in PBS was adjusted to a concentration of 50000 cells in 50 μl.

Ligands or combinations of ligands were added to the samples to a final concentration of 1 μM for TRH and 0.1 μM for MCP1, the natural ligands for the TRHR and CCR2 receptors, respectively. After the addition of the ligands, the cells were incubated at 37° C. for 10 min to allow the beta-arrestin-2-receptor interaction to occur. A Varian Eclipse fluorescence spectrometer (Varian) was used to record luminescence spectra in the range between 400-700 nm after coelenterazine h (Molecular Probes) was added to the samples to a final concentration of 5 μM.

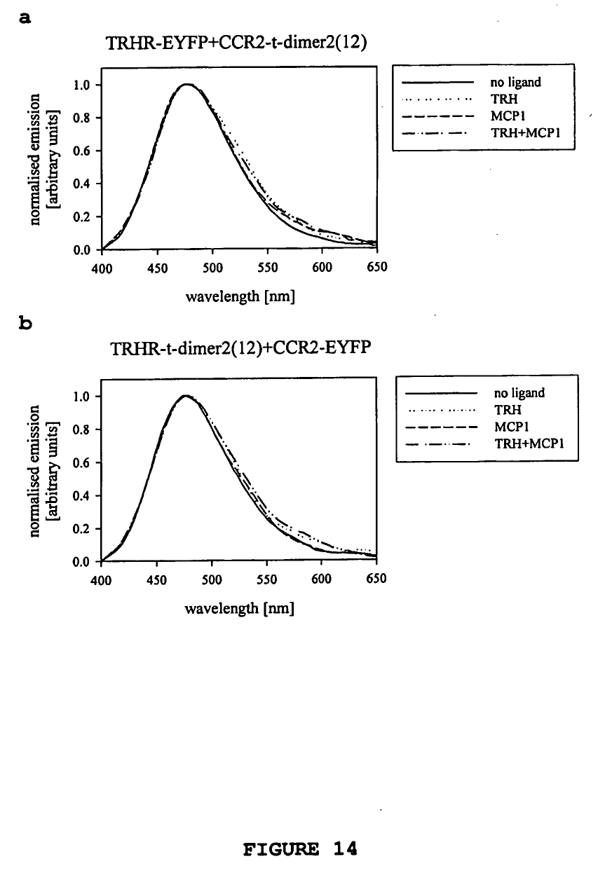

The luminescence spectra showed specific beta-arrestin-2-receptor interactions, depending on and selective for the receptor and its ligand (FIG. 14). If both ligands were added, both receptors associated with beta-arrestin-2, indicating the simultaneous activation of both receptors. This result was independent of which label was attached to which receptor and swapping the EYFP and t-dimer2(12) yielded essentially the same results (FIG. 14).

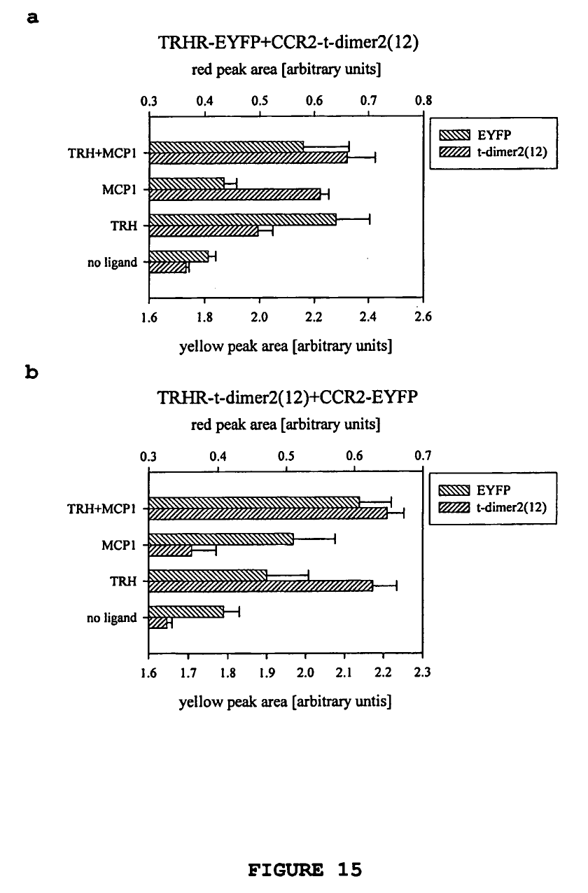

The spectra were further analysed by integrating the peak areas of the EYFP and t-dimer2(12) emission peaks as described in Example 2. The peak areas were clearly indicative of the presence of the ligands (FIG. 15), and also indicated that the interaction between TRHR and beta-arrestin-2 is stronger or in a more favourable orientation than it is with CCR2. An independent detection of both interactions was achieved as is required for a multiplex detection system.

Overall, this example demonstrates the multiplex detection of different protein-protein interactions in a dynamic, inducible system. Proteins involved range from membrane bound receptors to cytoplasmic proteins and detection can occur in live, mammalian cells.

### EXAMPLE 7 SIMPLIFIED DETECTION OF MOLECULAR ASSOCIATES

A simple detection system that yields a signal only if a specific complex molecular associate is formed, regardless of intermediate or other partial associates, would be very useful for some applications. As a model for the interactions within an associate of three components a fusion protein of three DTs was analysed.

The coding sequences of EGFP and Rluc were amplified via PCR with the following oligonucleotides (Table 3): EGFP-P2-fw/re, template: pEGFP-N1 (Clontech); Rluc-P3-fw/re, template phRL-CMV (Promega). The ends of the products were cut with the appropriate restriction enzymes and cloned into the vector pET-mRFP1 together with an oligonucleotide linker, encoding a peptide spacer between the subunits. This resulted in the following constructs: pET-mRFP1, pET-mRFP1-12-EGFP and pET-mRFP1-12-EGFP-Rluc (FIG. 4). The proteins were expressed in E. coli Rosetta cells and purified as described in Example 1.

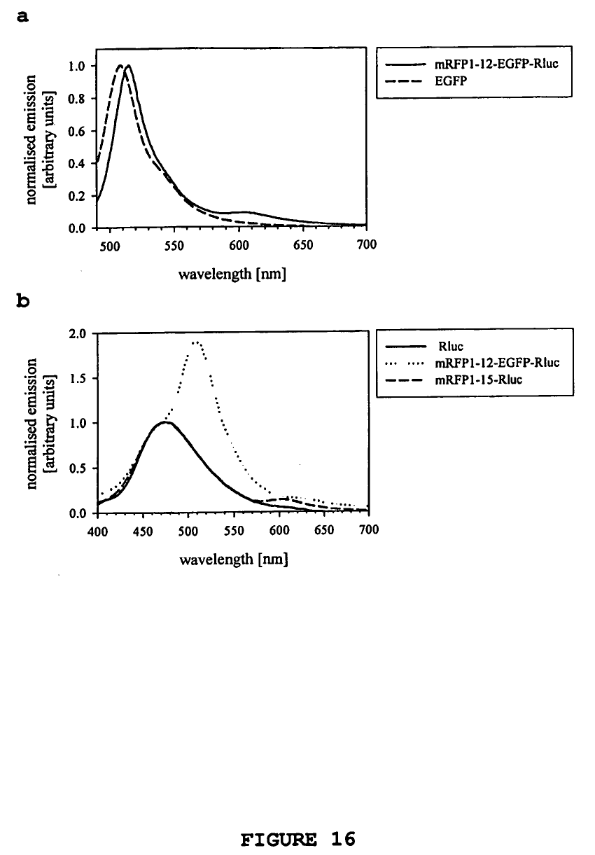

The FRET between EGFP and mRFP1 was analysed by exciting the mRFP1-12-EGFP-Rluc fusion protein with light at 480 nm. A FRET signal was detected after the mRFP1 emission due to direct excitation was subtracted as described in Example 1 (FIG. 16a). Next, the luminescence spectrum of mRFP1-12-EGFP-Rluc was recorded after the addition of 5 μM coelenterazine h (FIG. 16b). Rluc activated EGFP which in turn activated mRFP1 resulting in an activation of the green fluorescence at 510 nm as well as an increased activation of the red fluorescence between 600-650 nm. The observed mRFP1 fluorescence emission was higher compared to the mRFP1-15-Rluc construct which does not include an EGFP subunit.

Taken together, these result demonstrates that upon activation of Rluc (DT1) an increased mRFP1 (DT3) emission is detected when EGFP (DT2) is present at the same time within the associate, indicating the formation of a trimeric complex.

### EXAMPLE 8 INTERACTIONS IN COMPLEX MOLECULAR ASSOCIATES

The analysis of complex molecular associates containing more than two components requires a detection system that yields distinct signals for possible combinations of components within the associate. As a first model for the interactions within an associate of three components a fusion protein of three DTs was analysed.

The coding sequences of ECFP and EYFP were amplified via PCR with the following oligonucleotides (Table 3): EGFP-P3-fw/re, template: pECFP-N1 (Clontech); EGFP-P2-fw/re, template: pEYFP-N1 (Clontech). The ends of the products were cut with the appropriate restriction enzymes and cloned into the vector pET-mRFP1 together with an oligonucleotide linker, encoding a peptide spacer between the subunits. This resulted in the following constructs: pET-mRFP1-12-EYFP, pET-mRFP1-15-ECFP and pET-mRFP1-12-EYFP-ECFP (FIG. 4). The proteins were expressed in E. coli Rosetta cells and purified as described in Example 1.

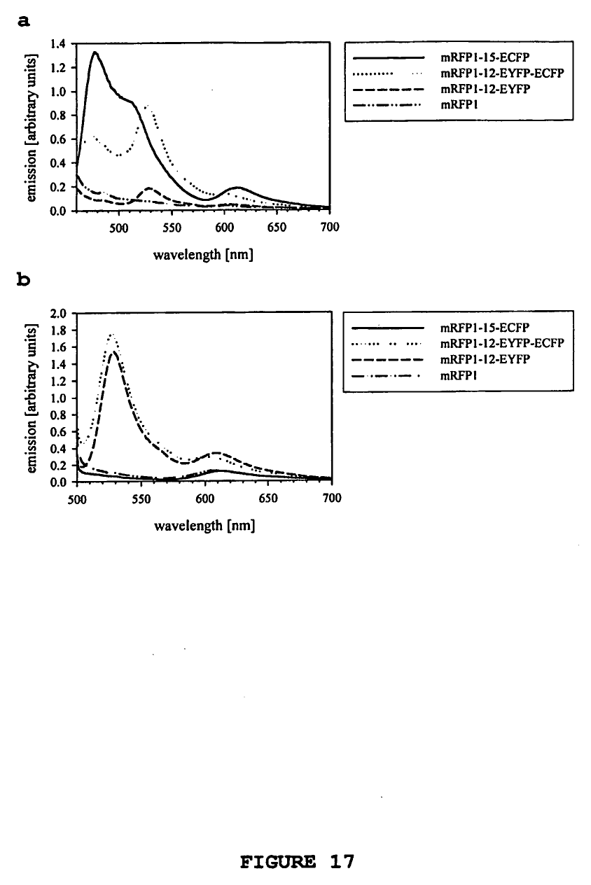

Excitation of the mRFP1-12-EYFP-ECFP construct at 440 nm resulted in emission from ECFP as well as EYFP and mRFP1 due to RET (FIG. 17a). RET also occurred from ECFP to mRFP1, in the absence of EYFP, using the mRFP1-15-ECFP construct. Without ECFP being present in the associate, EYFP and mRFP1 were not significantly activated by the 440 nm excitation light (FIG. 17a). Changing the excitation light to 490 nm activated EYFP and resulted in RET to mRFP1 in the constructs mRFP1-12-EYFP and mRFP1-12-EYFP-ECFP, while mRFP1 was not significantly activated in the absence of EYFP as was observed with mRFP1 and mRFP1-15-ECFP (FIG. 17b). Thus the sequential activation of DT1 (ECFP) and DT2 (EYFP) and detection of the emissions of all DTs provided accurate information on the composition of the molecular associate.

As a second model system for a complex molecular associate a combination of protein fusions and the high-affinity interaction between biotin and streptavidin was analysed.

Similar to Example 3, the coding sequence of the avitag was inserted into the construct pET-EYFP-ECFP as a linker consisting of the hybridised oligonucleotides AvitagN-fw/re (Table 3). The linker was cloned into position 1 (FIG. 4) of this construct. The coding sequence for biotin ligase was amplified with the oligonucleotides birA-fw/re using E. coli Top10 as a template. The birA gene was cloned into position 4 of the construct (FIG. 4) to co-express biotin ligase for a quantitative biotinylation of the of the avitag peptide sequence attached to the target protein. The resulting construct for the expression of a biotinylated EYFP-ECFP fusion protein was called pET-Avi-EYFP-ECFP/birA. The proteins were expressed in E. coli Rosetta cells and purified as described in Example 1.

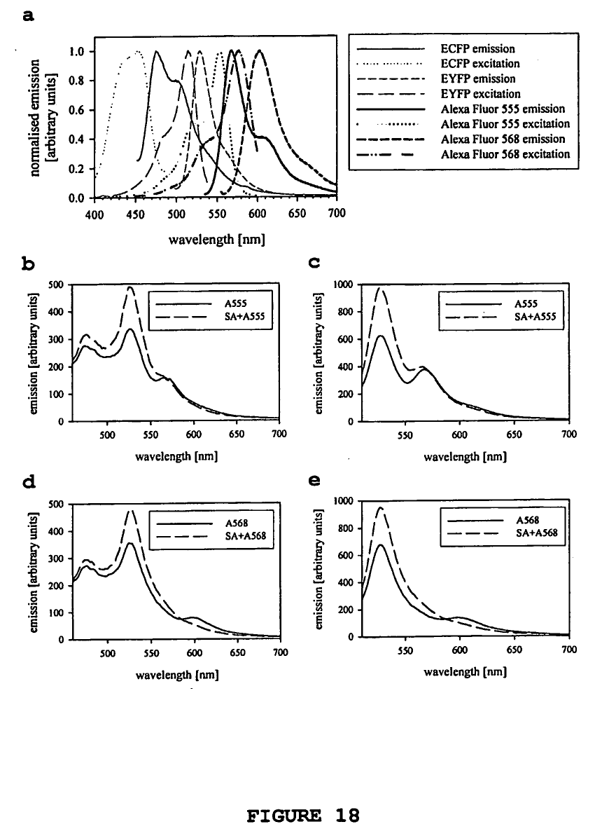

Alexa Fluor 555 and Alexa Fluor 568 conjugates were tested as DT3s as their excitation spectra overlapped well with the emission spectra of EYFP and ECFP (FIG. 18a). The biotinylated EYFP-ECFP fusion protein was mixed with roughly equal amounts of fluorescence conjugated streptavidins. As controls, the biotinylated EYFP-ECFP protein was preincubated with unconjugated, non-fluorescent streptavidin before adding in the streptavidin conjugate. Thus, the interaction between the fusion protein and the fluorescent streptavidin was blocked. The mixtures were excited at 440 nm, and the fluorescence emission was scanned. RET was observed from ECFP to EYFP and also to Alexa Fluor 555 or Alexa Fluor 568 (FIG. 18b,d). There was some emission from the Alexa Fluor 555 dye in the control reaction due to direct excitation of the dye by the light source. This emission however, was significantly smaller than the emission from the Alexa Fluor 555-streptavidin:EYFP-ECFP associate. Direct excitation of the Alexa Fluor 568 dye was not observed because of the greater spectral distance to the excitation light. When the same samples were excited at 490 nm, RET between EYFP and Alexa Fluor 555 or Alexa Fluor 568 was observed which was partly (Alexa Fluor 555) or completely (Alexa Fluor 568) blocked when the dye-streptavidin:EYFP-ECFP interaction was inhibited (FIG. 18c,e).

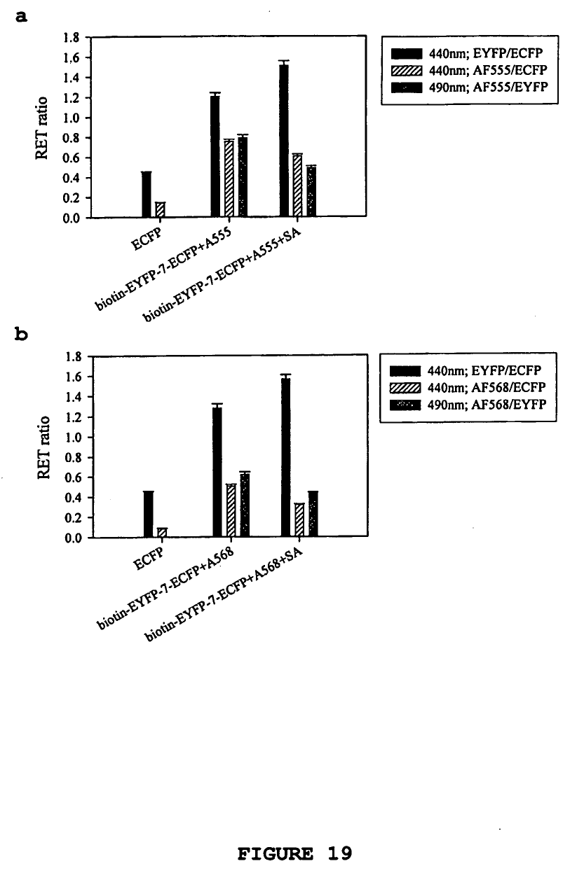

Next, RET ratios were calculated from the spectra in FIG. 18 b-e. The ratios provided further evidence that the detection system accurately determines the interactions within the molecular associate. RET ratios for the Alexa Fluor-streptavidin:EYFP-ECFP complex were significantly higher than in the controls where this interaction was blocked (FIG. 19). This was observed for RET between ECFP and Alexa Fluor (440 nm excitation) as well as RET between EYFP and Alexa Fluor (490 nm excitation). The ratios obtained with Alexa Fluor 555 were higher compared to the ratios with Alexa Fluor 568. However, the dynamic range and the signal-to-noise ratio were better with Alexa Fluor 568 because of the greater spectral separation and thus, lower background signal.

Taken together, this example demonstrates that 3 suitable DTs in combination with a sequential excitation of DT1 and DT2 and detection of light emission of DT1, DT2, and DT3 or DT2 and DT3, respectively, represents a system for the accurate detection of the composition of a complex molecular associate.

### EXAMPLE 9 DETECTION OF COMPLEX MOLECULAR ASSOCIATES IN MAMMALIAN CELLS

An important aspect of the function of G-protein coupled receptors (GPCRs) is their ability to form homo and hetero oligomeric complexes (see for example Kroeger et al., (2003), Frontiers Neuroendocrinol., 24: 254-278). In this example the assay system of the present invention was tested to detect various combinations of receptor-receptor complexes. This complex formation and the subsequent detection occurred in membranes of live, mammalian cells.

The cDNA encoding the fluorescent protein mRFP1 was amplified by PCR using the oligos mRFP1-fw/mRFP1-P3-re as described in Example 1. The PCR product was cloned into the plasmid pcDNA-MCS (Example 5) resulting in the vectors pcDNA-MCS-mRFP1 which was capable to express the fluorescent protein in mammalian cells. The PCR product containing the CCR2 cDNA sequence (Example 5) was cloned 5′ of mRFP1 into the plasmids pcDNA-MCS-mRFP1 to assemble an expression construct of a C-terminal fusion of the CCR2 receptor with mRFP1. The resulting final plasmid was named pcDNA-CCR2-mRFP1. Additionally, plasmids pcDNA-CCR2-ECFP and pcDNA-CCR2-EYFP from Example 5 were used here.

Cos-7 cells were transfected by one or a combination of the expression vectors using Genejuice (Novagen) transfection reagent according to the manufacturer's protocol and as described in Example 5. After 2 days incubation under standard culture conditions the cells were trypsinised, and the concentration of the cell suspension was adjusted to about 50000 cells in 50 μl PBS.

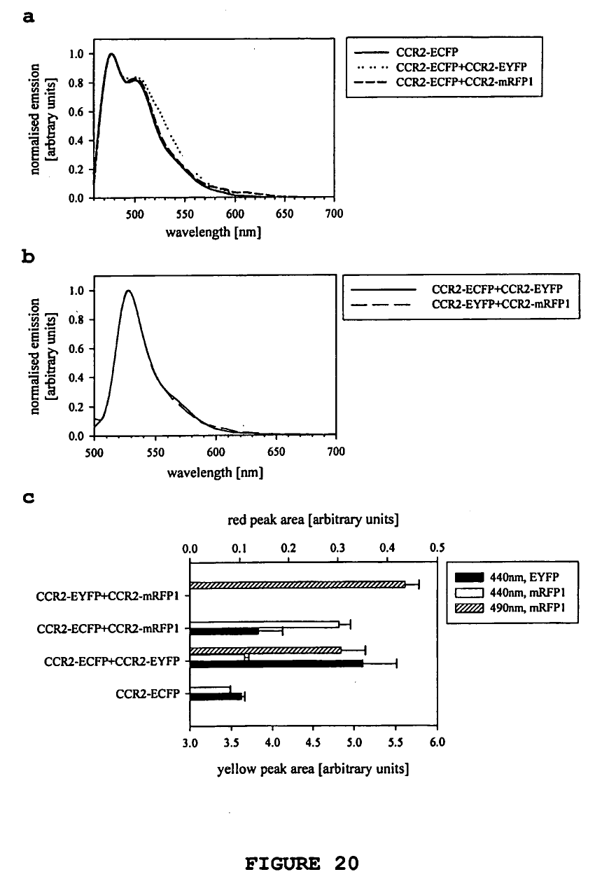

These cell suspensions were analysed by fluorescence spectroscopy using a Varian Eclipse fluorescence spectrometer (Varian). First, the samples were excited at 440 nm, the excitation maximum of ECFP, and the fluorescence emission was recorded between 460 and 700 nm. Then the excitation wavelength was changed to 490 nm, the excitation maximum of EYFP, and the fluorescence emission was recorded between 500 and 700 nm. All spectra were corrected for direct excitation of the acceptor fluorophore by the light source as described in Example 1. The spectra were further normalised to their emission maximum (arbitrary value “1”) (FIG. 20a, b). These spectra were also quantitated by integration of the respective emission peak areas as described in Example 2 (FIG. 20c).

RET was observed between ECFP-EYFP and ECFP-mRFP1 but mRFP1 was also a suitable energy acceptor for EYFP in this experiment as was indicated by an increase in the emission peaks. This was also confirmed by the integration of the respective peak areas which indicated a signal increase above background controls. Thus, the system was able to accurately detect all possible dimeric receptor complexes: CCR2-ECFP/CCR2-EYFP, CCR2-ECFP/CCR2-mRFP1 and CCR2-EYFP/CCR2/mRFP1.

This is different from the multiplex detection system illustrated by Examples 4-6 and the simple detection system described in Example 7; the first detects multiple interactions in parallel, the latter specifically detects a complex containing all components simultaneously. Neither is capable of detecting all possible pairwise associations as was demonstrated here (Examples 8+9).

In summary, this example demonstrates that this invention provides a system being capable of detecting dynamic, complex molecular associations in live mammalian cells. This can involve, but is obviously not restricted to, membrane bound proteins which are notoriously difficult to analyse.

1. A multi-component detection system comprising:
i). a first agent comprising a first interacting group coupled directly or indirectly to a first tag, which first tag emits light of a first wavelength upon activation by a substrate or energy source, which produces a first activated tag;
ii). a second agent comprising a second interacting group coupled directly or indirectly to a second tag, which second tag can accept the energy from the first tag when the first and second interacting groups are associated and an appropriate substrate or energy source for the first tag is present thereby producing a second activated tag that emits light of a second wavelength;
iii). a third agent comprising a third interacting group coupled directly or indirectly to a third tag that can accept the energy from the first activated tag when the first and third interacting groups are associated and an appropriate substrate or energy source for the first tag is present to produce a third activated tag that emits light of a third wavelength;
iv). an appropriate substrate or energy source to activate the first tag, and
v). a means of detecting said emitted light.

2. A multi-component detection system comprising:
i). a first agent comprising a first interacting group coupled directly or indirectly to a first tag, which first tag emits light of a first wavelength upon activation by a substrate or energy source which produces a first activated tag;
ii). a second agent comprising a second interacting group coupled directly or indirectly to a second tag, which second tag can accept the energy from the first tag in i) when the first and second interacting groups are associated and an appropriate substrate or energy source for the first tag in i) is present thereby producing a second activated tag that emits light of a second wavelength;
iii). a third agent comprising a third interacting group coupled directly or indirectly to a third tag that can accept the energy from the second activated tag in ii) when the first, second and third interacting groups are associated and an appropriate substrate or energy source for the first tag in i) is present to produce a third activated tag that emits light of a third wavelength, but said third tag is not substantially activated by the first activated tag in i) when only the first and third interacting groups are associated;
iv). an appropriate substrate or energy source to activate the tag in i); and
v). a means of detecting said emitted light.

3. A multi-component detection system comprising:
i). a first agent comprising a first interacting group coupled directly or indirectly to a first tag, which first tag emits light of a first wavelength upon activation by a substrate or energy source which produces a first activated tag;
ii). a second agent comprising a second interacting group coupled directly or indirectly to a second tag, which second tag can accept the energy from the first tag in i) when the first and second interacting groups are associated and an appropriate substrate or energy source for the first tag in i) is present thereby producing a second activated tag that emits light of a second wavelength;
iii). a third agent comprising a third interacting group coupled directly or indirectly to a third tag that can accept the energy from the first activated tag in i) when the first and third interacting groups are associated and an appropriate substrate or energy source for the first tag in i) is present and that can accept the energy from the second activated tag in ii) when the second and third interacting groups are associated and an appropriate substrate or energy source for the second tag in ii) is present to produce a third activated tag that emits light of a third wavelength;
iv). an appropriate substrate or energy source to activate the tags in i) and ii); and
v). a means of detecting said emitted light.

4. A multi-component detection system comprising:
i). a first agent comprising a first interacting group coupled directly or indirectly to a first tag, which first tag emits light of a first wavelength upon activation by a substrate or energy source which produces a first activated tag;
ii). a second agent comprising a second interacting group coupled directly or indirectly to a second tag, which second tag can accept the energy from the first tag in i) when the first and second interacting groups are associated and an appropriate substrate or energy source for the first tag in i) is present thereby producing a second activated tag that emits light of a second wavelength;
iii). a third agent comprising a third interacting group coupled directly or indirectly to a third tag consisting of a non-fluorescent quencher molecule that can accept the energy from:
a). the first activated tag when the first and third interacting groups are associated; and/or
b). the second activated tag when the second and third interacting groups are associated;
and an appropriate substrate or energy source for the first and/or second tag is present, whereby the light emission from the first and/or second activated tag is decreased;
iv). an appropriate substrate or energy source to activate the tags in i) and ii); and
v). a means of detecting said emitted light.

5. A multi-component detection system comprising:
i). a first agent comprising a first interacting group coupled directly or indirectly to a first tag, which first tag emits light of a first wavelength upon activation by a substrate or energy source which produces a first activated tag;
ii). a second agent comprising a second interacting group coupled directly or indirectly to a second tag, which second tag emits light of a second wavelength upon activation by a substrate or energy source, which produces a second activated tag;
iii). a third agent comprising a third interacting group coupled directly or indirectly to a third tag, which third tag can accept the energy from the first activated tag when the first and third interacting groups are associated and an appropriate substrate or energy source for the first tag is present to produce a third activated tag that emits light of a third wavelength;
iv). a fourth agent comprising a fourth interacting group coupled directly or indirectly to a fourth tag, which fourth tag can accept the energy from the second activated tag when the second and fourth interacting groups are associated and an appropriate substrate or energy source for the second tag is present to produce a fourth activated tag that emits light of a fourth wavelength;
v). an appropriate substrate or energy source to activate the first and second tags, and
vi). a means of detecting said emitted light.

6. A multi-component detection system comprising:
i). a first agent comprising a first interacting group coupled directly or indirectly to a first tag, which first tag emits light of a first wavelength upon activation by a substrate or energy source, which produces a first activated tag;
ii). a second agent comprising a second interacting group coupled directly or indirectly to a second tag, which second tag can accept the energy from the first tag when the first and second interacting groups are associated and an appropriate substrate or energy source for the first tag is present thereby producing a second activated tag that emits light of a second wavelength;
iii). one or more further agents comprising one or more further interacting groups coupled directly or indirectly to one or more further tags that can accept the energy from the first activated tag when the first and one or more further interacting groups are associated and an appropriate substrate or energy source for the first tag is present to produce one or more further activated tags that emit light of one or more further wavelengths, wherein said further wavelengths are different to the first or second wavelengths;
iv). an appropriate substrate or energy source to activate the first tag, and
v). a means of detecting said emitted light.

7. The system of claim 1, wherein the interacting group is selected from the group consisting a compounds, proteins, protein domains, protein loops, protein termini, peptides, hormones, protein-lipid complexes, lipids, carbohydrates, carbohydrate-containing compounds, nucleic acids, oligonucleotides, pharmaceutical agents, pharmaceutical drug targets, antibodies, antigenic substances, viruses, bacteria, and cells.

8. The system of claim 1, wherein the interacting group is selected from the group consisting of carbohydrates, proteins, drugs, chromophores, antigens, chelating compounds, molecular recognition complexes and combinations thereof.

9. The system of claim 7, wherein the nucleic acid molecule comprises DNA including single-stranded and double-stranded DNA, RNA including heterogeneous ribonucleic acid (hnRNA), transfer ribonucleic acid (tRNA), messenger ribonucleic acid (mRNA), or ribosomal ribonucleic acid (rRNA).

10. The system of claim 1, wherein external stimuli are applied to directly or indirectly modulate the association of interacting groups.

11. The system of claim 10, wherein the external stimuli are reagents comprising organic and inorganic molecules, proteins, nucleic acids, carbohydrates, lipids, ligands, drug compounds, agonists, antagonists, inverse agonists or compounds.

12. The system of claim 10, wherein the external stimuli are changes of conditions including temperature, ionic strength or pH.

13. The system of claim 1, wherein the detection tag is selected from the group consisting of a bioluminescent protein, a fluorescent protein, a fluorescent moiety or a non-fluorescent quencher.

14. The system of claim 13, wherein the bioluminescent protein is selected from the group consisting of luciferase, galactosidase, lactamase, peroxidase or any protein capable of luminescence in the presence of a suitable substrate.

15. The system of claim 13, wherein the fluorescent protein selected from the group consisting of green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz, GFPuv, destabilised EGFP (dEGFP), destabilised ECFP (dECFP), destabilised EYFP (dEYFP), HcRed, t-HcRed, DsRed, DsRed2, t-dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin or any other protein capable of fluorescence.

16. The system of claim 13, wherein the fluorescent moiety is selected from the group consisting of Alexa Fluor dyes and derivatives, Bodipy dyes and derivatives, Cy dyes and derivatives, fluorescein and derivatives, dansyl, umbelliferone, fluorescent and luminescent microspheres, fluorescent nanocrystals, Marina Blue, Cascade Blue, Cascade Yellow, Pacific Blue, Oregon Green and derivatives, Tetramethylrhodamine and derivatives, Rhodamine and derivatives, Texas Red and derivatives, rare earth element chelates or any combination or derivative thereof or any other molecule with fluorescent properties.

17. The system of claim 13, wherein the non-fluorescent quencher is selected from the group consisting of dabcyl, non-fluorescent pocilloporins, QSY-7, QSY-9, QSY-21, QSY-35, BHQ-1, BHQ-2, BHQ-3 or any known non-fluorescent chromophore with the ability to absorb light and to quench fluorescence and/or luminescence.

18. The system of claim 1, wherein the activation energy is generated by a light source including lasers, Hg-lamps, Xe-lamps and halogen lamps or any light emitting device, and the generated light is restricted to a specific wavelength or wavelength range by appropriate means including laser lines, bandpass filters, shortpass filters, monochromators or any device to spectrally restrict the emitted light.

19. The system of claim 1, wherein the first activation energy is generated from a suitable enzyme substrate selected from the group consisting of coelenterazine and luciferin or a derivative derived therefrom.

20. The system of claim 3, wherein sequentially the first detection tag is activated and the light emitted from the first, second and third detection tag is detected and then the second detection tag is activated and the light emitted from the second and third detection tag is detected.

21. The system of claim 4, wherein sequentially the first detection tag is activated and the light emitted from the first, second detection tag is detected and then the second detection tag is activated and the light emitted from the second detection tag is detected.

22. The system of claim 20, wherein the sequence of activation and detection is repeated over time to yield temporal information.

23. The system of claim 1, wherein the interacting group and tag are coded for in a fusion protein construct.

24. A recombinant DNA encoding the fusion protein construct of claim 23.

25. A fusion gene that comprises the recombinant DNA of claim 24.

26. A DNA cassette comprising a promoter operably linked to one or more fusion protein genes of claim 23.

27. A vector comprising the fusion gene of claim 25.

28. A host cell transiently or stably transformed or transfected by a vector of claim 27.

29. The host cell of claim 28, wherein the cell is human, mammalian, insect, plant, bacterial, or yeast.

30. The vector of claim 27, wherein the gene construct is under the control of a constitutive promoter.

31. The vector of claim 27, wherein the gene construct is under the control of an inducible promoter.

32. The vector of claim 30, wherein the constitutive promoter is selected from the group consisting of CMV, SV40, RSV, EF-1 a, Tk, and AdML, when the cell to be transformed or transfected is mammalian.

33. The vector of claim 30, wherein the constitutive promoter is selected from the group consisting of T7, AraBAD, trc, pL, tac, and lac, when the cell to be transformed or transfected is a bacterial cell.

34. The vector of claim 30, wherein the constitutive promoter is selected from the group consisting of nmtl, gall, gallO, TEF1, AOX1, GAP, and ADH1, when the cell to be transformed or transfected is a yeast cell.

35. A virus comprising the fusion gene of claim 25.

36. A host cell infected by the virus of claim 35.

37.-45. (canceled)
